(19) United States
(12) Patent Application Publication
(10) Pub. No.: US 2011/0097798 A1
(43) Pub. Date: Apr. 28, 2011

(54) MAMMALIAN CELL EXPRESSION VECTORS AND UTILIZATION

(75) Inventors: Qiang LI, Edmonton (CA); Xing-Zhen Chen, Edmonton (CA)

(73) Assignee: ATGCELL INC., Edmonton, AB (CA)

(21) Appl. No.: 12/995,909

(22) PCT Filed: Jun. 30, 2009
(86) PCT No.: PCT/CA2009/000908

§ 371 Date: Jan. 14, 2011

Related U.S. Application Data
(60) Provisional application No. 61/076,987, filed on Jun. 30, 2008.

(51) Int. Cl. C12N 5/02; C12N 15/63

(52) U.S. Cl. 435/375; 435/320.1

(57) ABSTRACT

An improved mammalian expression vector system which allows: (1) highly expressing exogenous proteins in host mammalian cells; (2) rapidly and efficiently screening recombinant stable cell lines expressing the gene of interest; (3) maintain sustainable expression and prevent gene silencing; and (4) effectively secreting proteins into media in some of cases. The entire expression vector system includes optimized promoters and core promoters, the use of special internal ribosome entry sites, integration of the bacterial backbone into the mammalian expression unit, multiple choices of selection markers, artificial matrix attachment region elements, effective secreting lead sequences and their 5′ and 3′ UTRs, and proper combinations of these expression elements.

### FIELD OF THE INVENTION

The present invention relates to the field of exogenous gene expression in mammalian cells and efficient selection of high-producing clones.

### BACKGROUND

Recombinant protein expression systems are based on the introduction of a foreign gene in an expression vector into prokaryotic or eukaryotic cells, as an additional episome or integrated part of the host cell genome. The production of foreign proteins is then achieved by efficient transcription and translation by host cell machineries. Commonly used hosts are bacterial, yeast, insect and mammalian cells. Of these, mammalian expression systems enable the production of recombinant proteins that possess relevant post-translational modifications and exhibit high enzymatic activity. They are therefore superior to and more suitable than those using cells of lower species for the production of proteins for research and therapeutic purposes.

Recombinant protein expression is an important technique to produce useful proteins for vaccine development, therapeutics, diagnostics, gene therapy, crystallography, and studies of biochemical/physiological functions. Many therapeutic proteins are marketed products and many more are in clinical development. These therapeutic proteins are currently used to relieve patients suffering from many disease conditions, including various cancers, heart attacks, strokes, cystic fibrosis, diabetes, anaemia, haemophilia, Gaucher's disease and include proteins such as insulin, interleukins, interferons, growth hormones, erythropoietin, tissue plasminogen activator, therapeutic monoclonal antibodies, and the like. However, future growth depends largely on whether the industry can overcome a number of hurdles, including drug delivery challenges and cost issues.

Advances in mammalian cell culture and recombinant DNA technologies have facilitated the expression of a variety of therapeutic proteins using genetically engineered cells. The expression of many biologically active therapeutic proteins, which are derived from higher eukaryotic sources, often requires specific post-translational modifications which do not naturally occur in prokaryotic and lower eukaryotic cells, thus necessitating the use of cells derived from higher eukaryotic sources. For example, the expression of glycoproteins in mammalian cells has the advantage of providing proteins which contain natural glycosylation. Glycoproteins produced from mammalian sources contain outer chain carbohydrate moieties, which are markedly different from those counterparts produced from lower eukaryotes. The use of mammalian cells as hosts for production of secreted mammalian proteins has the significant advantage over the use of lower eukaryotes, because mammalian cells have a secretory system that readily recognizes and properly processes secretion-directed proteins.

For efficient expression of a target protein in mammalian cells, well-designed expression vectors, appropriate host cells and efficient gene transfer methods are important factors. In the last two decades, a variety of expression vectors have been developed, which allows propagating and expressing covalently linked genes in different types of host cells. Although many cultured mammalian cells of different origins can be used for this purpose, only a few are suitable for large-scale production. Good gene transfer methods will permit the introduction of a target gene into host cells for improved transient or stable expression. There are also many gene transfer reagents commercially available that affect expression efficiency.

Manufacturing recombinant proteins on an industrial scale requires technologies that can engineer stable and high-producing cell lines rapidly, reproducibly and with relative ease. Identifying stable and high-producing transfectants is normally laborious and time consuming.

A mammalian cell expression vector is generally a plasmid that is used to introduce and express a specific gene in a host cell, allowing transcription of large amounts of stable mRNAs, which will be translated into proteins. Once the expression vector is inside the cell, the encoded protein is produced by the cellular transcription and translation machineries as well as necessary post-translational modifications. Therefore, expression vectors are basic tools for biotechnology and production of proteins. Development of optimally designed vectors is critical for achieving safe and efficient exogenous gene expression in transfected cells.

In the last two decades, much effort has been made to improve gene transfer vectors, both viral and non-viral vectors. It is now known that the expression level of an introduced gene depends mostly on the strength of transcriptional regulatory elements and the transduction efficiency of the gene transfer vector. In eukaryotic cells, transcription of protein-coding genes by RNA polymerase II is mediated by numerous factors, including sequence-specific DNA-binding proteins, transcriptional coregulators, chromatin-remodeling factors, enzymes that covalently modify histones, and other proteins.

In eukaryotic cells, translation of cellular mRNA is generally mediated by a “cap” at the 5′ end of mRNA upstream of the coding sequence in the untranslated region (UTR) which is responsible for interaction of the messenger RNA with the ribosome. However, certain viruses, including picornaviruses, rhinoviruses and Hepatitis C viruses, have been shown to possess an upstream region, designated Internal Ribosome Entry Sites (IRES), which mediates translation in the absence of the cap structure at the 5′ end of the messenger RNA. These IRES are defined as dicistronic mRNAs. A nucleotide sequence functions as an IRES if it can direct translation of the second cistron in a manner that is independent of the first cistron, when present in the intercistronic region of a dicistronic mRNA. This IRES region is typically at least 450 nucleotides long in viruses, and is located within the 5′ UTR and downstream from the cap. Certain cellular mRNAs, such as those encoding BiP, c-myc, and eIF4G also contain IRES elements. How these IRES elements assist in mediating translation is not well known.

Foot and mouth disease virus (FMDV) IRES has been reported to enable the translation of two open reading frames from one mRNA with high levels of expression (1). Recently, the 196 nucleotide 5′ UTR of mouse GTX mRNA was found to contain a 9-nucleotide segment that can function as an IRES element. Because this stretch of 9 nucleotides is complementary to 18S rRNA, it was suggested that IRES activity was due to the ability of the 9-nucleotide segment to base-pair with rRNA, thereby recruiting the ribosome to the RNA (2, 3). Furthermore, it was reported that introduction of specific mutations into this synthetic IBES derived from the GTX homeodomain protein revealed additional transcriptional activity (4).

The availability of selectable markers suitable for use in mammalian cells has permitted analysis of the influence of the stable expression of single or multiple genes on host cells. Several antibiotic-based selectable marker genes and respective selection reagents are available, including marker gene NEO with selection reagent geneticin, HYG from Streptomyces hygroscopicus with hygromycin B, ZEO (or Sh ble) from Staphylococcus aureus with zeocin or phleomycin, PUR from Streptomyces alboniger with puromycin. There are also amplifiable selective marker genes, including dihydrofolate reductase (DHFR) and glutamine synthetase (GS). The former is based on the dhfr gene coding for the DHFR enzyme, which catalyses the conversion of dihydrofolate to tetrahydrofolate. Methotrexate (MTX) inhibits the DHFR enzyme, but DHFR deficient host cells that transfected an expression vector containing the dhfr gene can develop resistance by amplifying the dhfr gene. As the amplification unit is much larger than the size of dhfr gene, genes of interest that are located in the same vector, are co-amplified. GS is the enzyme responsible for the biosynthesis of glutamine from glutamate and ammonium. This enzymatic reaction provides the only pathway for glutamine formation in a mammalian cell. Therefore in the absence of glutamine in the growth medium, the GS enzyme is essential for the survival of the mammalian cells in culture. For cell lines which do not express sufficient endogenous GS, a transfected GS gene can function as a selectable marker by permitting growth in a glutamine-free medium. For cell lines which do contain sufficient active GS, the specific GS inhibitor, methionine sulphoximine (MSX), can be used to inhibit endogenous GS activity such that only transfectants with additional GS activity can survive. Recently, fluorescent proteins, such as green fluorescent protein (GFP) or HcRED, can also be used as selectable markers, through flow cytometry process.

Matrix Attachment Regions (MARs) or Scaffold Attachment Regions (SARs) are non-consensus-like AT-rich DNA elements which are several hundred base pairs in length, and which organize the nuclear DNA of the eukaryotic genome into some 60,000 chromatin domains, 4-200 kbp loops, by periodic attachment to the protein scaffold or matrix of the cell nucleus. MARs have been isolated from regions surrounding actively transcribed genes but also from introns, centromeres and teleomeric regions and have been found to collaborate with enhancers to help regulate transcription by controlling the chromatin state of DNA. MARs positively interact with enhancers, form loop domains and often are located at the borders of transcriptionally active domains. This has led to the idea of using MARs as flanking elements around transgenes, forming so-called mini-domains, in order to protect transgenes or expression cassettes from transcriptional silencing and the effects of surrounding heterochromatin (transcriptionally inactive chromatin), as well as to possibly increase gene expression. Several reports have shown that a MAR in a flanking position can strongly stimulate expression of a transgene, as well as reduce expression variability among cell clones. Moreover, due to the character of a mini-domain, expression should be independent of the integration site. Eukaryotic chromatin are typically arranged as 50-200 kb chromosomal loop domains whose bases are attached to the intranuclear matrix by different non-histone proteins, including nuclear lamin, topoisomerase and components of the transcription and RNA-processing machineries. Structurally, MARs usually comprise AT-rich sequences of high unwinding propensity and enforce a curved DNA structure (5, 6).

### SUMMARY OF THE INVENTION

The present invention provides an expression vector system that allows rapid and effective screening of high producing recombinant cell strains in a manner that provides an effective combination of such properties as production yield, screening efficiency, time line, and cost. In particular, the vectors of the present invention provide an improved combination of expression levels and the time required for strain isolation.

The present invention concerns the addition or modification of specific cis-acting DNA elements to expression plasmids that function as enhancement elements in order to increase expression level.

In one embodiment, synthetic AT-rich MAR-like DNA fragments protect expression cassettes from transcriptional silencing and the effects of surrounding heterochromatin, as well as to possibly increase gene expression.

A novel cell clone selection approach is utilized by fusing different type(s) selection marker genes into a composite gene to maximize selection efficiency.

The present invention utilizes a concept that minimizes or shortens the size of expression vectors in order to reduce unnecessary burden on host mammalian cells.

The present use of HEK and CHO cells can require up to 6-9 months achieving optimized levels of recombinant protein expression. In contrast, the expression systems of the present invention can reach similar levels of expression in significantly less time, typically 6-9 weeks. Stated differently, a procedure that entails screening on the order of 1000 clones using conventional techniques may be accomplished by screening on the order of only 100 clones by the presently claimed invention.

In one aspect, the invention comprises a vector structure useful for selection and expression of a target cDNA in mammalian cells. The vector comprises the following elements, preferably, but not necessarily, in the order provided:
a) a first artificial MAR element;
b) a strong composite promoter and core promoter sequence for initiating and transcribing the target cDNA in mammalian cells;
c) a cDNA sequence encoding a protein product of choice;
d) a synthetic GTX IRES;
e) a promoter enabling expression of a single or composite selective marker gene;
f) a single or composite selective marker gene;
g) a terminator sequence; and
h) a second artificial MAR element.

In one embodiment, the vector comprises a composite selective marker gene only, such as ZEO-PUR, ZEO-HYG, ZEO-GFP, DHFR-ZEO-PUR, DHFR-ZEO-HYG, DHFR-ZEO-GFP under the control of GTX IRES.

In another embodiment, the vector comprises two separate selection marker genes: a first selective marker gene X such as GFP, HcRED, PURO, ZEO, DHFR or GS under the controlled of GTX IRES, and a second selective marker gene Y such as HYG, ZEO, ZEO-PURO, ZEO-GFP under the control of FMDV IRES.

In one embodiment, the selective gene promoter comprises a bacterial promoter EM7 enabling expression of a single or composite selective marker gene in E. coli.

In one embodiment, the terminator sequence comprises a SV40 or bovine growth hormone (BGH) terminator sequence.

In one embodiment, the vector further comprises a PUC origin for high-copy amplification in E. coli.

The target gene is driven by the composite promoter and the selective marker sequence is driven by IRES so as to exhibit an activity level substantially below that of its corresponding wild type, which reduces the efficiency of translation initiation for the selection marker relative to that of the target gene, and allows preferential selection of cells expressing high levels of the protein of interest.

MAR elements can significantly help maintaining persistent expression of the target gene.

The invention also comprises a method of using a vector of the type described herein, the method comprising the steps of transfecting a mammalian cell population with a vector that comprises a target cDNA, a selective marker, a composite promoter controlling target cDNA and a weak IRES promoter controlling a selective marker. In one embodiment, actual expression level of selective marker protein is substantially lower that of target protein. Thus, surviving clones under high selection pressure are most likely to express high levels of the target protein.

Multiple selection options are created for rapid and efficient pick-up of high producing clones, including antibiotics selection of hygromycin, zeocin or purimycin; metabolic selection and amplification of DHFR or GS, and flow cytometry selection of GFP or HcRED.

Any reluctant gene sequence which is not crucial for expression in host mammalian cells is deleted. Selection of vectors in E. coli is partially built into mammalian cell selection system by inserting a bacterial EM7 promoter before the selective marker gene, other than bacterial PUC replication origin. Thus, the entire expression vector has only single expression cassette.

### BRIEF DESCRIPTION OF THE DRAWINGS

In the drawings, different expression elements are described in details as below. The drawings emphasize the principles of the present invention and are not necessarily to scale. Additionally, each of the embodiments depicted is one of possible arrangements utilizing the fundamental concepts of the present invention. The drawings are briefly described as follows:

FIG. 1. Comparison of different promoters in transient expression experiments. HEK, CHO, HELA and A549 cells are cultured in a 24-well plate in standard serum-containing media. Transfection of expression vectors containing different promoters (CMV, SFH, CAG, CEFR and EFHT) was conducted using Lipofactamine 2000 when cells grew to 60-70% confluency. Cell lysates were collected for Western blotting 72 hours post-transfection. The experiments have been repeated at least twice.

FIG. 2. Comparison of different promoters in stable expression experiments. HEK and CHO cells were cultured in a 24-well plate in standard serum-containing media. Transfection of expression vectors containing different promoters (CMV, SFH, CAG, CEFR and EFHT) was conducted using Lipofactamine 2000 when cells grew to 60-70% confluency. Stably expressed cells (mixed population, no single colony was picked) were selected in G418 (500-800 mg/L) containing media for 3 weeks. Cell lysates were collected for Western blotting after culture for another 3 weeks. The experiments have been repeated at least twice.

FIG. 3. Comparison of different core promoters in transient expression experiments. HEK and CHO cells were cultured in a 24-well plate in standard serum-containing media. Transfection of vectors containing different core promoters (C0-C3) inserted in CAG or SFH promoter was conducted using Lipofactamine 2000 when cells grew to 60-70% confluency. Cell lysates were collected for Western blotting 72 hours post-transfection. The experiments have been repeated at least twice.

FIG. 4. Some living cell examples in transient expression experiments. HELA cells were cultured in a 24-well plate in standard serum-containing media. Transfection of vectors containing different promoter sequences (upper left; CMV-C2; upper right, SFH-C2; bottom left: CAG-C2; bottom right: EFHT-C2) was conducted using Lipofactamine 2000 when cells grew to 60-70% confluency. Living cell images were taken 72 hours post-transfection.

FIG. 5. Some living cell examples in stable expression experiments. MDCK cells were cultured in a 24-well plate in standard serum-containing media. Transfection of vectors containing different promoter sequences (upper left; CMV-C2; upper right, SFH-C2; bottom left: CAG-C2; bottom right: EFHT-C2) was conducted using Lipofactamine 2000 when cells grew to 60-70% confluency. Stably expressed cells (mixed population, no single colony was picked) were selected in G418 (500-800 mg/L) containing media for 3 weeks. Living cell images were taken immediately after the end of antibiotic selection.

FIG. 6 shows schematic representations of REEMAC expression vector schemes.

FIG. 7. Expression comparison between the first-version vector REEMAC1 (1) and the second-version vector REEMAC2 (2). REEMAC vectors hosting human interferon-α1 fused with an Fc tag were transiently transfected (upper panel) and stably transfected (lower panel) in CHO-K1 cells. Expressed proteins were collected from the culture medium and probed with an anti-Fc antibody in a Western blotting. The difference is that REEMAC2 do not have the second expression cassette for antibiotic selection marker.

FIG. 8. Expression stability analysis among different REEMAC vectors (1. REEMAC2; 2. REEMAC3Q; 3. REEMAC3R). Human interferon α1 fused with an Fc tag was stably expressed in HEK or CHO cells. Stable cell line pools were cultured for over a period of time, as indicated. Expressed proteins were collected from the culture medium and probed with an anti-Fc antibody in a Western blotting. We have designed 18 artificial MAR fragments, which are inserted before the promoter. We finally found that fragment R is the best one for preventing from silencing and Q is the second best one.

FIG. 9. Effect of 5′ and 3′ UTRs on protein secretion. There are significant difference for interferon α1 and P53 in term of protein secretion efficiency.

FIG. 10. Sample proteins stably expressed in HEK cells. All genes were cloned into REEMAC1 vector and fused with an Fc tag on their C-termini. The collected medium supernatant were resolved in 8% SDS-PAGE and detected with an anti-Fc antibody. Chemokine family: 1 CCL2; 2 CCL3; 3 CCL4; 4 CXCL1; 5 CXCL2; 6 CXCL4; 7 CCL3; Interleukin family (IL): 8 IL5; 9 IL7; 10 IL8; Interferon family (IFN): 11 IFN-α1; 12 IFN-α4; Cell surface antigens: 13. CD40; 14 CD54; Cytokine receptors: 15 Tumor necrosis factor receptor type 1; 16 Tumor necrosis factor receptor type 2; 17 Epithelial growth factor receptor; 18 Nerve growth factor receptor; Reporter gene: 19 green fluorescent protein (GFP); Transcription factor: 20 P53; Protein kinases: 21 MAP kinase-3; 22 Protein kinase A α-subunit; 23 Cyclin-dependent kinase-2.

FIG. 11. Purified sample proteins. The proteins (interferon-α1, chemokine CXCL1 and interleukin-5, CD8A and CD44) were fused with an Fc tag and expressed in HEK cells and purified using protein A column from the culture media. The proteins were resolved in 8% SDS-PAGE and stained with Coomassie blue.

### DETAILED DESCRIPTION OF PREFERRED EMBODIMENTS

The present invention relates to a novel vector structure useful for selecting and expressing a target cDNA in a mammalian cell system, as well as methods of transfecting mammalian cells using the vectors described herein and methods of producing exogenous proteins from cultured mammalian cells. Any term or expression not expressly defined herein shall have its commonly accepted definition understood by those skilled in the art. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The following are specific examples of embodiments of the present invention. These examples demonstrate how the expression vector systems of the present invention can be used in establishing high-producing stable cell lines in various host mammalian cells within a relatively short period of time, and how target genes can be regulated by sequences within such vectors. These examples are offered by way of illustration and are not intended to limit the invention in any manner.

An expression cassette is made up of one or more genes and the sequences controlling their expression. A single expression cassette comprises a unique transcription unit that drives the expression of the gene of interest and the selectable marker through an internal ribosome entry site (IRES). This gene expression system ensures that stable clones express the gene of interest.

A composite promoter is a gene promoter which combines the enhancer, promoter core and 5′ untranslated region (UTR).

A selective marker is a marker will protect the organism from a selective agent that would otherwise kill it or prevent its growth. A composite selective marker is a chimeric marker containing two or more selective marker genes.

Generally, in this system, the target cDNA and the selective marker gene or genes are expressed in a single mammalian expression cassette under the control of a strong composite promoter, and the two or three separate genes are linked by IRES elements. Thus, the target transcript is highly expressed and the selective marker transcript(s) are weakened. Without restriction to a theory, it is believed that the weakened activity of the marker protein allows the rapid identification and isolation of cell lines. This is particularly true in cells where the vector has been incorporated into regions of particularly high transcriptional activity. It is also believed that the high rate of transcription is able to compensate for the weakened marker activity, in order to provide detectable levels of the marker.

In one embodiment, the general vector scheme comprises:
a) A first artificial MAR element, such as for example MAR-R;
b) A strong composite promoter (such as for example CAG or SFH) and a core promoter (such as for example C2) initiating and transcribing the target cDNA;
c) A target cDNA encoding a protein product of choice;
d) a synthetic GTX IRES;
e) a bacterial promoter EM7 enabling expression of a single or composite selective marker gene in E. coli;
f) a single or composite selective marker gene;
g) A terminator such as SV40 terminator or bovine growth hormone (BGH);
h) optionally, a second artificial MAR element (such as for example MAR-S); and
i) PUC origin for high-copy amplification in bacteria.

The order of these elements may be varied. For example, some elements can be placed in other sites, such as the first or second MARs.

In one embodiment, optionally, there are two selective marker genes, X and Y, with a synthetic GTX IRES controlling selective marker gene X (for example, GFP, HIRED, DHFR, GS, PURO or ZEO) and a FMDV IRES controlling selective marker gene Y (for example, HYG, ZEO-PURO, ZEO-GFP). FMDV IRES/selective marker gene Y may follow GTX IRES/selective marker gene X.

In one embodiment, the bacterial promoter EM7 is located between GTX IRES and the single or composite selective marker gene. In this case, EM7 can drive the constitutive expression of either of antibiotic resistance gene in E. coli for isolating correct plasmids. All bacterial backbone except the replication origin of the vector may be deleted from the expression vector to avoid repression of target protein expression in mammalian cells by bacterial elements. Furthermore, reduced vector size will also increase gene transfection efficiency and facilitate its integration into host cell genome.

In one embodiment, selected artificial MARs can protect expression cassettes from transcriptional silencing by insulating the effects of surrounding transcriptionally inactive heterochromatin. MARs also strongly stimulate expression of transgenes as well as reduce expression variability between cell clones.

Optimization of Promoters/Enhancers

Viral promoters have widely been used to achieve high levels of heterologous expression in cultured cells. Only a few are considered ubiquitously strong promoters, which includes cytomegalovirus major immediate-early promoter/enhancer (CMV), simian virus 40 promoter (SV40), Rous sarcoma virus genome long terminal repeat promoter (RSV), moloney murine leukemia virus long terminal repeat promoter (MLU), and mouse myeloproliferative virus long terminal repeat promoter (MPSV) (7-9). Of these, CMV is active in many cell culture systems as it is considered one of the strongest promoters, and thus is so far the most frequently used for in vitro and in vivo studies (10). Our previous studies as well as those by many others (11-13) demonstrated that the target protein expression driven by CMV is gradually attenuated over time in cultured cells. Other viral promoters have not been reported to exhibit such expression attenuation but have lower promoter activities than that of CMV (14-16).

It was reported that combined use of CMV and other regulatory elements allows long-term expression in transfected cells, suggesting that the expression attenuation driven by the CMV promoter can be prevented or alleviated by other transcriptionally active elements. Some CMV hybrid promoters are able to increase expression level and persistence, e.g., a CMV promoter plus its intron A or human elongation factor 1 alpha (EF1α) intron 1; composite promoters composed of a CMV enhancer, chicken beta-actin promoter plus its intron 1; or a CMV enhancer and ubiquitin B promoter plus its intron (17-20).

Natural cellular promoters, in particular, mammalian promoters, possess several advantages over viral promoters, for example, they allow expression of a protein of interest under more physiological conditions and exhibit more uniform expression level. Furthermore, mammalian promoters are less prone to inactivation than viral promoters (21).

In one embodiment, the following five promoters which are believed to represent the strongest ones were tested for their capability of driving exogenous expression: (I) human CMV promoter; (II) SFH composite promoter (SV40 enhancer, human ferritin heavy chain core promoter and the human T-cell leukemia virus 5′ UTR); (III) CAG composite promoter (CMV enhancer, chicken beta-actin promoter plus its intron 1); (IV) CEFR composite promoter (CMV enhancer, the elongation factor EF1α core promoter and the human T-cell leukemia virus 5′ UTR); and (V) EFHT composite promoter (human elongation factor EF1α core promoter and the human T-cell leukemia virus 5′ UTR).

In one embodiment, the vector backbone is from pEGFPC2 (Clontech), where human CMV promoter drives Green Fluorescent Protein (GFP) reporter gene expression. Using different strategies, we successfully substituted CMV promoter with each of CAG, SFH, CEFR and EFHT composite promoters.

We transiently transfected the same amount of these five plasmids into several types of host mammalian cells, including HEK, CHO, HELA and A549. We measured their expression levels 72 hours post-transfection by Western blotting and/or real-time monitored by fluorescence microscopy. Furthermore, we generated HEK and CHO stable cell line pools (selection for 6 weeks) and evaluated their expression levels in a similar way.

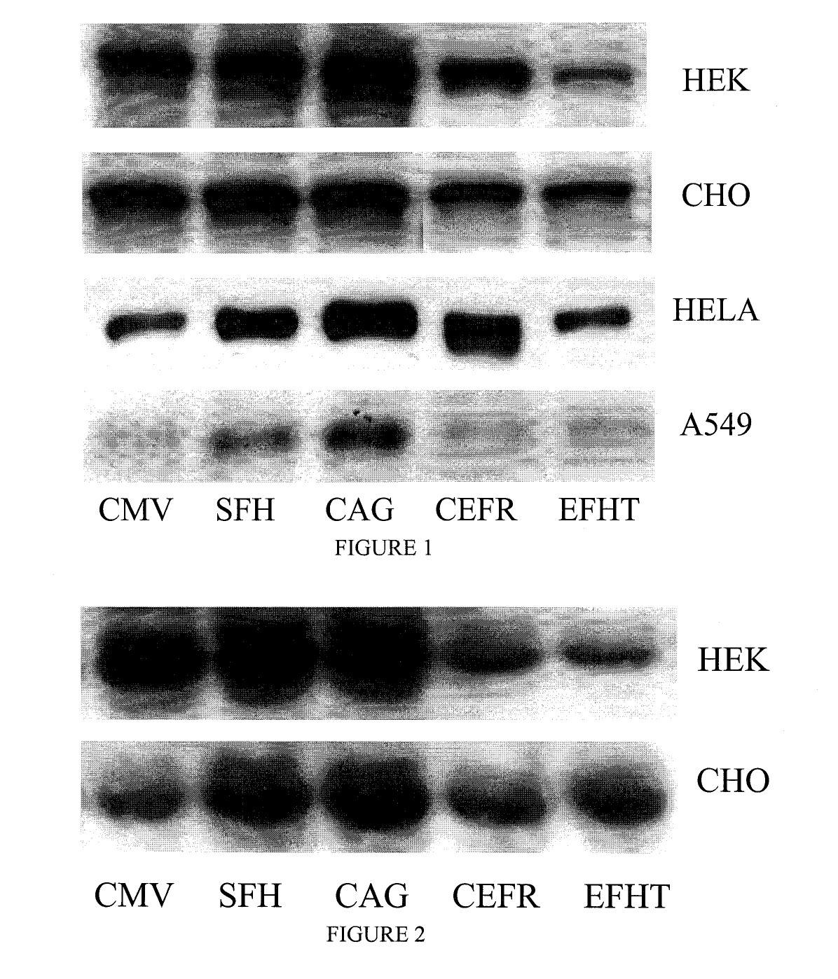

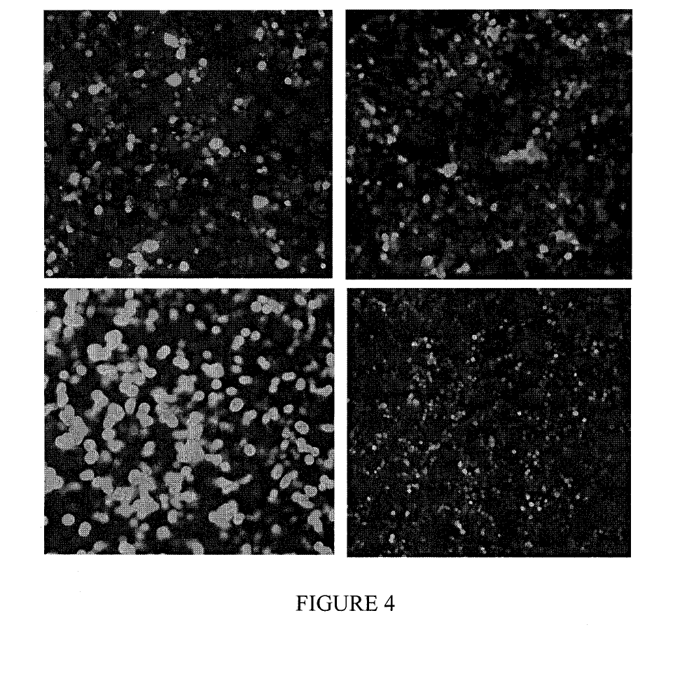

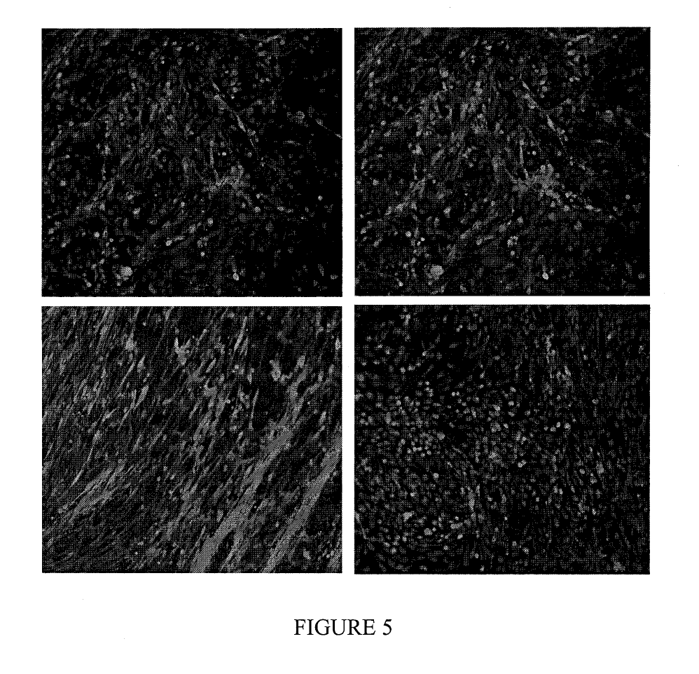

We found that CAG and SFH promoters gave the highest expression levels in transient and stable transfection experiments (FIGS. 1-2). Daily observation for GFP signal intensity under fluorescence microscopy were also conducted, which gave similar results as Western blotting (see representative images in FIGS. 4-5). CAG was the strongest promoter we tested in most host cells. SFH is the designed composite promoter and proved to be a very good promoter (better than CMV). Therefore, preferred embodiments of the present invention utilize either the CAG or SFH promoters.

Embodiments of promoter sequences are provided in Appendix A, attached hereto.

### Optimization of Core Promoter Elements

Every eukaryotic gene has a core promoter that resides at the extreme 5′ end of its transcription unit. It encompasses the transcription start site (TSS) and typically extends ˜35 nt either upstream or downstream of TSS. A key function of the core promoter is to direct the initiation of transcription by the basal RNA polymerase II machinery. The core promoter is the ultimate target of action of many sequence-specific transcription factors and coregulators (22). There are several core promoter elements, including the TATA box (consensus: T-A-T-A-A-A; position: −25 to −30 nt) for binding the TATA box-binding protein subunit of the TFIID complex, transcription factor IIB recognition element (BRE; G/C-G/C-G/A-C-G-C-C; with the 3′ C of BRE immediately followed by the 5′ T of the TATA box) for binding the TFIIB complex, initiator (Inr; Py-Py(C)-A(+1)-N-T/A-Py-Py) for cooperatively binding TFIID, downstream core promoter element (DPE; +28 to +32 nt; A/G-G-A/T-C/T-G/A/C) for binding TFIID in coordination with Inr, and motif ten element (MTE; +18 to +29 nt; C-G/C-A-G/A-C-G/C-G/C-A-A-C-G-G/C) that modulates interaction of TFIID with the core promoter (23-28). These core promoter elements are not universally present in all core promoters. Rather, each element is found only in a subset of core promoters. Several combinations of more than one core promoter elements were found to be synergistically advantageous for transcription initiation: e.g., TATA box with Inr, DPE with Inr, BRE with TATA box, MTE with TATA box, and MTE with DPE (29, 30). Statistical data also showed that most human gene promoters have at least one core promoter element at a functional position. In many cases, the presence of a synergetic combination of two elements, which is much stronger than a single element, dictates the position of TSS (31, 32). Recently, it was shown that DPE and MTE together enhance transcription when added to the heterologous core promoter (33-35).

We tested the following combinations of core promoter elements in transient transfection experiments to evaluate their expression levels. The C0 core promoter was originally used in the expression vector. We substituted C0 to C1, C2 or C3 core promoter in CAG or SFH promoter setting.

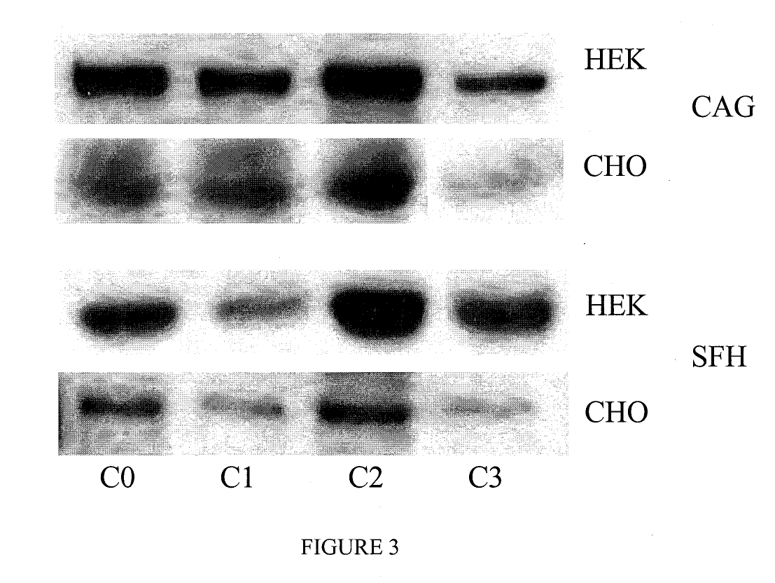

We found that the C2 core promoter gave the highest expression (FIG. 3). The core promoter sequences are provided in Appendix B, attached hereto.

Introduction of IRES into the Expression Vector

When selectable markers are used to identify host mammalian cells that have stably integrated the vector elements into their genome, they are essentially being used as surrogates to identify cells that express the target protein. However, the linked integration of the marker gene and the target gene is not always guaranteed, and it is possible that cells which exhibit drug-resistant phenotypes or survive in nutrient deficient media may not express the target protein. In general, target and selectable marker genes were introduced in one or more independent expression cassette plasmids. Although this approach can be used successfully, there is an increased risk of producing drug-resistant colonies that do not co-express the target protein, thus, in one embodiment; the vector utilizes secondary screening strategies.

In order to increase the likelihood of cells overexpressing high levels of both the target protein and selection marker, the present invention comprises a dicistronic expression vector where the target gene, driven by a strong composite promoter, is followed by an IRES, such as viral FMDV IRES or synthetic GTX IRES and inserted in front of the marker gene. These elements allow ribosome binding and cap-independent initiation of marker gene translation. However, since the translation of genes downstream of IRES often is not very efficient, these dicistronic vectors minimize marker gene synthesis while insuring the linkage of integrating the target gene and selection marker.

In this strategy, bicistronic expression vectors allow rapid and efficient selection of positive clones that express target proteins. They include a single expression cassette that includes the target gene together with a selection marker or reporter gene from the same promoter, so that virtually all transfected cells expressing the selection marker also express the gene of interest. With these vectors, fewer colonies need to be screened to identify clones expressing high levels of the target protein. Ribosomes can enter the bicistronic mRNA either at the 5′ end to translate the gene of interest, or at FMDV IRES or GTX IRES to translate the selection marker or a reporter gene, which allows preferential selection of cells highly expressing the target proteins. The IRES elements may drive selection marker gene(s) expression in a weakened manner Sequences for the exemplary FMDV or GTX IRES are provided in Appendix C, attached hereto.

Integration of Most of the Bacterial Backbone Unit into the Mammalian Expression Unit

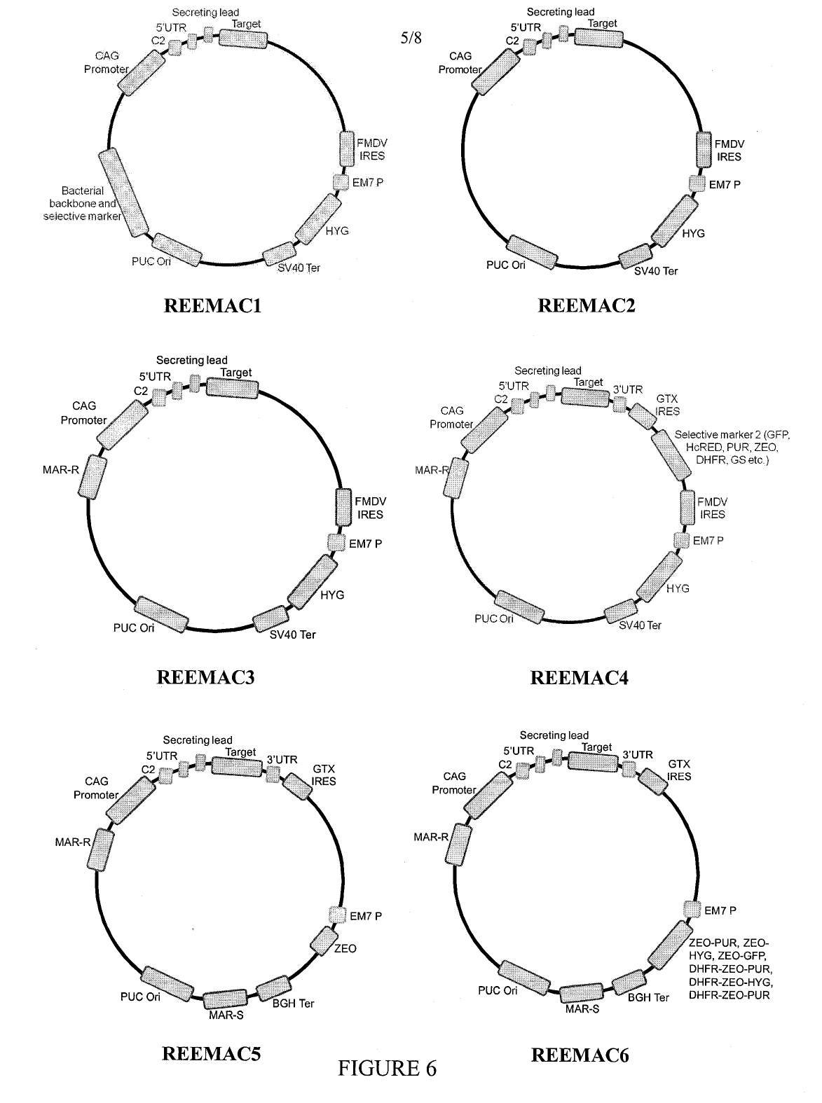

Recent studies showed that covalent linkage of bacterial DNA to the mammalian expression cassette results in transcriptional silencing in host cells (36-39). We believe that expression cassette free of bacterial DNA sequences in an active vector form is at least partially responsible for high and persistent expression in cell lines stably overexpressing target proteins. Thus, in one embodiment, the DNA components in both the mammalian expression cassette unit and the bacterial backbone unit are systematically altered. As shown in FIG. 6, a bacterial expression promoter EM7 may be provided between the IRES element and selection marker gene, which allows selecting/growing the plasmid in bacteria (REEMAC1). Bacterial expression elements were deleted and all other optional elements which are not crucial for expression in mammalian cells (REEMAC2 and afterward) were also deleted. In such a setting, E. coli high-copy replication origin PUC on was re-orientated outside the mammalian expression cassette. With this arrangement, the vector size was significantly reduced (to about 2 kb) and possible effect of expression repression of bacterial backbone on mammalian cells was eliminated.

EM7 Promoter Sequence (66 nt):

TTGACAATTAATCATCGGCATAGTATATCGGCATAGTATAATACGACT
CACTATAGGAGGGCCACC

Therefore, in one embodiment, the present invention comprises the integration of the bacterial backbone into the mammalian expression unit to reduce vector size and eliminate possible repressive effect on target gene expression in host cells.

### Selection Marker Genes

A selection marker is required to identify and select cells that have integrated the plasmid into the host genome. Moreover, by physically linking the expression of the marker and target protein, and using selection strategies that specifically impair the ability to express the marker relative to the target protein, it is possible to stringently select cells expressing high levels of the target protein.

Reports from other groups and our own experiments demonstrated that some cell lines continue to be naturally resistant to certain markers, making direct selection difficult or less efficient. For example, non-transfected MDCK cells can tolerate up to 300 μg/ml G418 and resistant MDCK cell lines with tolerance to higher G418 doses can be generated in 10 to 14 days during stable cell line selection. Thus, the characterization of other cell selection markers is of great interest. We examined effects of different selection reagents and/or combinations on parental host cells to identify which marker genes are suitable for efficient selection. In one embodiment, HYG, ZEO or PUR are preferred selection reagents in most of cell lines tested.

In one embodiment, the invention comprises novel dual or triple selection markers utilizing IRES, which allows rapid and efficient selection of host stable cell lines. DHFR or GS amplifiable selective marker(s) (alone or fused with other selective marker gene) serve as the secondary selection method in one embodiment. Alternatively, GFP or HcRED reporter gene can replace or fuse with one of the marker genes. In this latter case, positive clones were enriched by GFP-based flow cytometry sorting (REEMAC4 series).

### Artificial Matrix Attachment Region Elements in the Expression Vector

Cell lines stably expressing target proteins are typically generated by transfection followed by integration of the expression plasmid into the host genome via nonhomologous recombination. Such integration is a random event that generates clones with a wide range of expression levels, reflecting the copy number of the integrated gene and the transcriptional activity of the locus in which the copies were integrated (positional effect). The concept that high expression loci exist in the genome has lead to the development of several strategies for targeting gene integration specifically to these transcriptionally active loci, thus introducing a controlled integration event.

The expression of recombinant proteins in mammalian cells is presently hampered by the high variation of transgene expression between individual cell clones. Variability of expression stems in part from epigenetic events linked to the packaging of the DNA into repressive chromatin structures, and/or from topological constraints resulting from the organization of chromosomes within the nucleus. MAR genetic elements have previously been proposed to insulate transgenes from repressive effects linked to their site of integration within the host cell genome. We have evaluated these elements in various stable transfection settings to increase the production of recombinant proteins. Using the GFP coding sequence, we found that that MAR elements mediate a dual effect on the population of transfected cells. First, MAR elements almost fully abolish the occurrence of cell clones that express little target gene (which may result from gene integration in an unfavorable chromosomal environment). Second, they increase the overall expression level of the target gene over large range of expression levels, allowing the identification of cells with significantly higher levels of expression.

The present invention comprises at least one artificial MAR fragment, which is preferably inserted before the promoter. In one embodiment, the artificial MAR fragment may comprise one of three specific MARs, each of which can maintain high protein expression levels over a long period of time (FIG. 8). Sequence information for specific embodiments of artificial MARs are provided in Appendix D, attached hereto.

### Secreting Lead Sequence and 5′ and 3′ UTRs

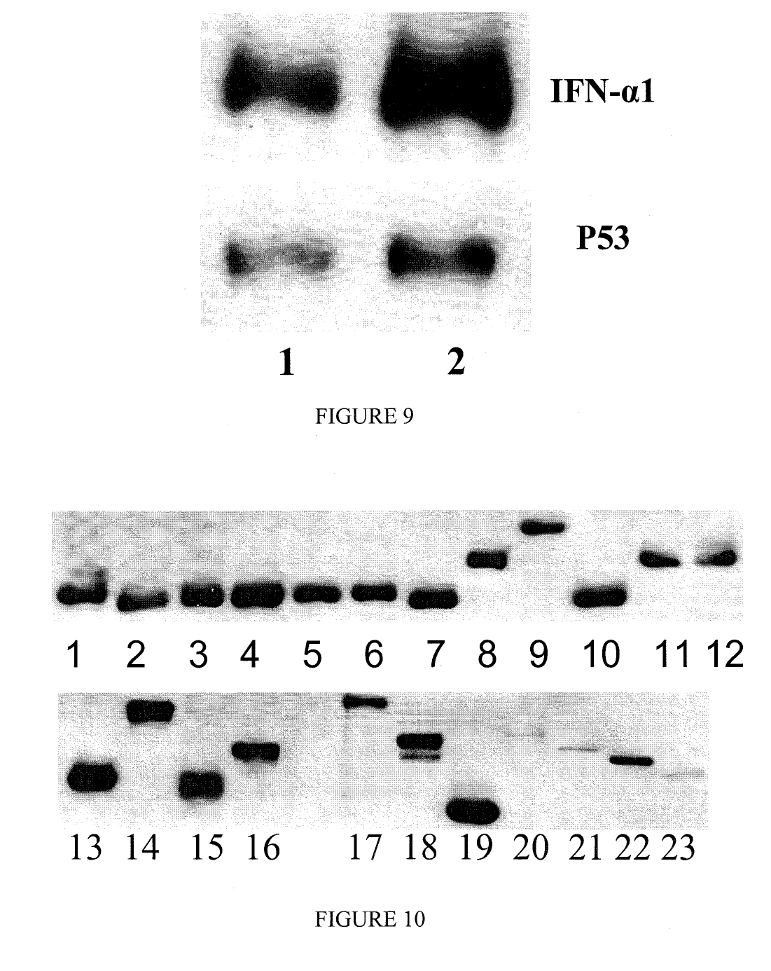

For secreted protein expression, secreting lead sequence and 5′ and 3′ UTRs are also important. In one embodiment, the invention comprises a secreting lead sequence and a UTR to enhance protein secretion (FIG. 9). In specific embodiments, the secreting lead sequence may comprise one of the following three sequences:

1. Murine Ig kappa chain v-j2-c (AAH80787)
lead peptide:
METDTLLLWVLLLWVPGSTGD (21aa)
2. Human CD33 (NP_001763) lead peptide:
MPLLLLLPLLWAGALAM (17aa)
3. Murine VH102 germline J558.33 (AF303864)
lead sequence:
MGWSCIILFLVATATGVHS (19aa)

In another embodiment, the UTR sequence may comprise one of the following two sequences: 5′ complete Kodak sequence: GCCGCCACCATGG or 3′ UTR of murine Ig kappa chain v-j2-c (M35669, 30 bp): AACGGGCTGATGCTGCACCAACTGTATCCA

### EXAMPLES

The invention will be further described with reference to the following non-limiting examples.

### Example 1

### Preparation of Gene Constructs

A large number of plasmid constructions are required in this invention. We initially used expression vector pEGFPC2 (Clontech) as a template. Different new vector elements, including promoters, core promoters, IRES, MARs, were either through in vitro oligo/gene synthesis or amplified from commercially available vectors or human genomic/cDNA library. Different subcloning strategies, such as regular PCR, reverse PCR, site-directed mutagenesis, restriction digestion and direct primer annealing, were used to integrate new elements into specific sites of a vector. Ligation, transformation and colony screening followed standard protocols. The authenticity of all constructs were confirmed by sequencing.

### Example 2

### Cell Culture and Transfection

Different types of host mammalian cells were cultured in standard conditions except indicated otherwise. Gene transfection was conducted using Lipofectamine 2000 or LTX (Invitrogen) according to the manufacturer's recommendation.

### Example 3

### SDS-PAGE and Immunoblotting

Different protein samples are resolved by SDS-PAGE (6-15% acrylamide depending on the target protein size) and transferred to nitrocellulose membranes. The membranes are blocked with 3% skim milk powder in 50 mM Tris, pH 8.0, 150 mM NaCl, 0.1% Tween 20, incubated with a specific primary antibody, and a horseradish-peroxidase-coupled secondary antibody. Blots are visualized and quantified using enhanced chemiluminescence reagent (GE Healthcare) and a Kodak Image Station. The used blots can be stripped off using Re-blot plus WB recycling kit (Chemicon) and re-probed with other antibodies up to 5 times.

### Example 4

FIG. 1 shows a comparison of different promoters in transient expression experiments. HEK, CHO, HELA and A549 cells were cultured in a 24-well plate in standard serum-containing media. Transfection of expression vectors containing different promoters (CMV, SFH, CAG, CEFR and EFHT) was conducted using Lipofactamine 2000 when cells grew to 60-70% confluency. Cell lysates were collected for Western blotting 48 hours post-transfection. The experiments were repeated at least twice.

### Example 5

FIG. 2 shows a comparison of different promoters in stable expression experiments. HEK and CHO cells were cultured in a 24-well plate in standard serum-containing media. Transfection of expression vectors containing different promoters (CMV, SFH, CAG, CEFR and EFHT) was conducted using Lipofactamine 2000 when cells grew to 60-70% confluency. Stably expressed cells (mixed population, no single colony was picked) were selected in G418 (500-800 mg/L) containing media for 3 weeks. Cell lysates were collected for Western blotting after culture for another 3 weeks. The experiments were repeated at least twice.

### Example 6

FIG. 3 shows a comparison of different core promoters in transient expression experiments. HEK and CHO cells were cultured in a 24-well plate in standard serum-containing media. Transfection of vectors containing different core promoters (C0-C3) inserted in CAG or SFH promoter was conducted using Lipofactamine 2000 when cells grew to 60-70% confluency. Cell lysates were collected for Western blotting 48 hours post-transfection. The experiments were repeated at least twice.

### Example 7

FIG. 4 shows examples of living cells in transient expression experiments. HELA cells were cultured in a 24-well plate in standard serum-containing media. Transfection of vectors containing different promoter sequences (upper left; CMV-C2; upper right, SFH-C2; bottom left: CAG-C2; bottom right: EFHT-C2) was conducted using Lipofactamine 2000 when cells grew to 60-70% confluency. Living cell images were taken 72 hours post-transfection.

### Example 8

FIG. 5 shows examples of living cells in stable expression experiments. MDCK cells were cultured in a 24-well plate in standard serum-containing media. Transfection of vectors containing different promoter sequences (upper left; CMV-C2; upper right, SFH-C2; bottom left: CAG-C2; bottom right: EFHT-C2) was conducted using Lipofactamine 2000 when cells grew to 60-70% confluency. Stably expressed cells (mixed population, no single colony was picked) were selected in G418 (500-800 mg/L) containing media for 3 weeks. Living cell images were taken immediately after the end of antibiotic selection.

### Example 9

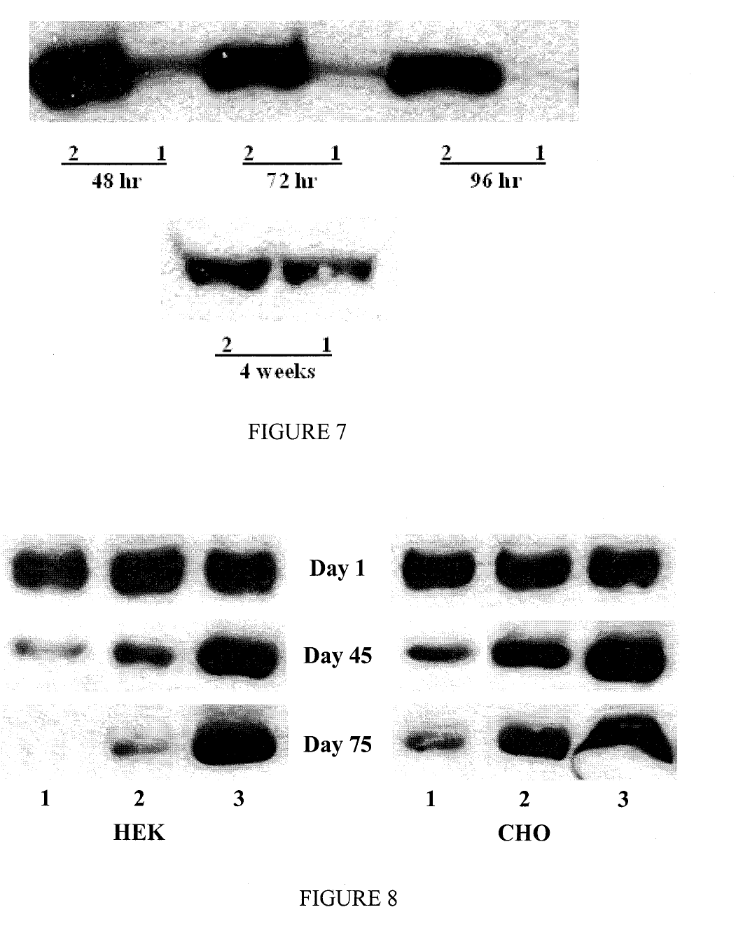

FIG. 6 shows REEMAC expression vector schemes. FIG. 7 shows expression comparison between the first-version vector REEMAC1 (1) and the second-version vector REEMAC2 (2). REEMAC vectors hosting human interferon-α1 fused with an Fc tag were transiently transfected (upper panel) and stably transfected (lower panel) in CHO-K1 cells. Expressed proteins were collected from the culture medium and probed with an anti-Fc antibody in a Western blotting. The difference is that REEMAC2 do not have the second expression cassette for antibiotic selection marker.

FIG. 8 shows expression stability analysis among different REEMAC vectors (1. REEMAC2; 2. REEMAC3Q; 3. REEMAC3R). Human interferon α1 fused with a Fc tag was stably expressed in HEK or CHO cells. Stable cell line pools were cultured for a period of time, as indicated. Expressed proteins were collected from the culture medium and probed with an anti-Fc antibody in a Western blotting. We have designed 18 artificial MAR fragments, which are inserted before the promoter. We finally found that fragment R is the best one for preventing from silencing and that Q is the second best.

### Example 10

FIG. 9 shows the effect of 5′ and 3′ UTRs on protein secretion. There are significant difference for interferon α1 and P53 in term of protein secretion efficiency.

### Example 11

FIG. 10 shows sample proteins stably expressed in HEK cells. All genes were cloned into the REEMAC1 vector and fused with a Fc tag on their C-termini. The collected medium supernatant were resolved in 8% SDS-PAGE and detected with an anti-Fc antibody. Chemokine family: 1 CCL2; 2 CCL3; 3 CCL4; 4 CXCL1; 5 CXCL2; 6 CXCL4; 7 CCL3; Interleukin family (IL): 8 IL5; 9 IL7; 10 IL8; Interferon family (IFN): 11 IFN-α1; 12 IFN-α4; Cell surface antigens: 13. CD40; 14 CD54; Cytokine receptors: 15 Tumor necrosis factor receptor type 1; 16 Tumor necrosis factor receptor type 2; 17 Epithelial growth factor receptor; 18 Nerve growth factor receptor; Reporter gene: 19 green fluorescent protein (GFP); Transcription factor: 20 P53; Protein kinases: 21 MAP kinase-3; 22 Protein kinase A α-subunit; 23 Cyclin-dependent kinase-2.

### Example 12

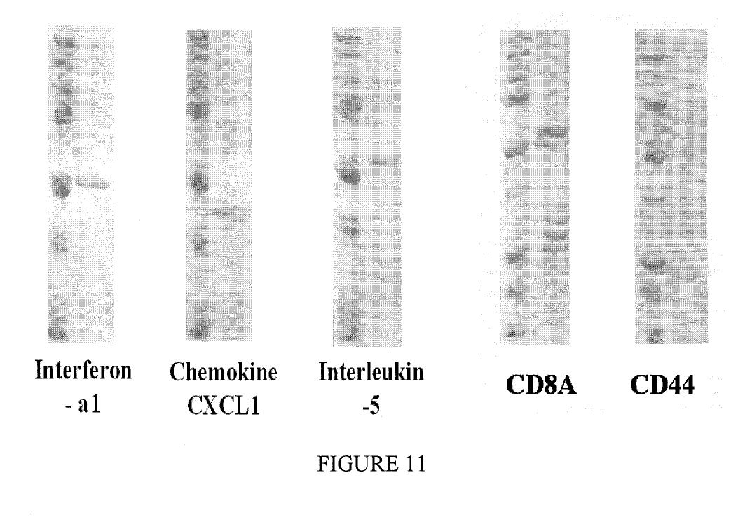

FIG. 11 shows purified sample proteins. The proteins (interferon-α1, chemokine CXCL1 and interleukin-5, CD8A and CD44) were fused with a Fc tag and expressed in HEK cells and purified using protein A column from the culture media. The proteins were resolved in 8% SDS-PAGE and stained with Coomassie blue.

### Example 12

TABLE 1
Expression levels of sample proteins stably expressed in HEK and
CHO cells. All genes were cloned into REEMAC1 vector and fused
with an Fc tag on their C-termini.

| | Yield in HEK Cells | Yield in CHO Cells |
|---|---|---|
| Protein Name | (pg/cell/day) | (pg/cell/day) |
| Chemokine CCL2 | 115 | 101 |
| Chemokine CCL4 | 89 | 55 |
| Chemokine CCL5 | 50 | 49 |
| Chemokine CXCL1 | 97 | 54 |
| Chemokine CXCL2 | 84 | 38 |
| Chemokine CXCL4 | 93 | 28 |
| Chemokine CXCL5 | 90 | 23 |
| Chemokine CXCL7 | 80 | 36 |
| Interleukine-2 | 86 | 27 |
| Interleukine-5 | 124 | 29 |
| Interleukine-8 | 107 | 27 |
| Interferon-α1 | 80 | 24 |
| Interferon-α4 | 118 | 20 |
| Insulin-like growth factor-2 | 102 | 37 |
| Nerve growth factor | 31 | 45 |
| Bone morphogenetic protein-2 | 35 | 8 |
| Transforming growth factor- | 18 | 23 |
| β1 P53 | 20 | 4 |
| Cyclin-dependent kinase-2 | 20 | 8 |
| Human monoclonal antibody | 45 | 37 |
| 1 | | |
| Mouse monoclonal antibody 1 | 43 | 31 |
| Rabbit monoclonal antibody 1 | 40 | 32 |

As will be apparent to those skilled in the art, various modifications, adaptations and variations of the foregoing specific disclosure can be made without departing from the scope of the invention claimed herein. The various features and elements of the described invention may be combined in a manner different from the combinations described or claimed herein, without departing from the scope of the invention.

### REFERENCES

The following references, listed above, are incorporated herein by reference, as if duplicated in their entirety (where permitted).
1. Ramesh, N., Kim, S. T., Wei, M. Q., Khalighi, M., Osborne, W. R. (1996) High-titer bicistronic retroviral vectors employing foot-and-mouth disease virus internal ribosome entry site. Nucleic Acids Res., 24, 2697-2700.
2. Chappell, S. A., Edelman, G. M., Mauro, V. P. (2000) A 9-nt segment of a cellular mRNA can function as an internal ribosome entry site (IRES) and when present in linked multiple copies greatly enhances IRES activity. Proc. Natl. Acad. Sci. U.S.A, 97, 1536-1541.
3. Chappell, S. A., Edelman, G. M., Mauro, V. P. (2004) Biochemical and functional analysis of a 9-nt RNA sequence that affects translation efficiency in eukaryotic cells. Proc. Natl. Acad. Sci. U.S.A, 101, 9590-9594.
4. Hartenbach, S., Fussenegger, M. (2006) A novel synthetic mammalian promoter derived from an internal ribosome entry site. Biotechnol. Bioeng., 95, 547-559.
5. Jenke, A. C., Stehle, I. M., Hellmann, F., Eisenberger, T., Baiker, A., Bode, J., Fackelmayer, F. O., Lipps, H. J. (2004) Nuclear scaffold/matrix attached region modules linked to a transcription unit are sufficient for replication and maintenance of a mammalian episome. Proc. Natl. Acad. Sci. U.S.A, 101, 11322-11327.
6. Bode, J., Winkelmann, S., Gotze, S., Spiker, S., Tsutsui, K., Bi, C., A K P, Benham, C. (2006) Correlations between scaffold/matrix attachment region (S/MAR) binding activity and DNA duplex destabilization energy. J. Mol Biol., 358, 597-613.
7. Xu, Z. L., Mizuguchi, H., Ishii-Watabe, A., Uchida, E., Mayumi, T., Hayakawa, T. (2001) Optimization of transcriptional regulatory elements for constructing plasmid vectors. Gene., 272, 149-156.
8. Xia, W., Bringmann, P., McClary, J., Jones, P. P., Manzana, W., Zhu, Y., Wang, S., Liu, Y., Harvey, S., Madlansacay, M. R., et al. (2006) High levels of protein expression using different mammalian CMV promoters in several cell lines. Protein Expr. Purif., 45, 115-124.
9. Al-Dosari, M., Zhang, G., Knapp, J. E., Liu, D. (2006) Evaluation of viral and mammalian promoters for driving transgene expression in mouse liver. Biochem. Biophys. Res. Commun., 339, 673-678.
10. Foecking, M. K., Hofstetter, H. (1986) Powerful and versatile enhancer-promoter unit for mammalian expression vectors. Gene., 45, 101-105.
11. Chen, W. Y., Bailey, E. C., McCune, S. L., Dong, J. Y., Townes, T. M. (1997) Reactivation of silenced, virally transduced genes by inhibitors of histone deacetylase. Proc. Natl. Acad. Sci. U.S.A., 94, 5798-5803.
12. Grassi, G., Maccaroni, P., Meyer, R., Kaiser, H., D'Ambrosio, E., Pascale, E., Grassi, M., Kuhn, A., Di Nardo, P., Kandolf, R., Kupper, J. H. (2003) Inhibitors of DNA methylation and histone deacetylation activate cytomegalovirus promoter-controlled reporter gene expression in human glioblastoma cell line U87. Carcinogenesis., 24, 1625-1635.
13. Choi, K. H., Basma, H., Singh, J., Cheng, P. W. (2005) Activation of CMV promoter-controlled glycosyltransferase and beta-galactosidase glycogenes by butyrate, tricostatin A, and 5-aza-2′-deoxycytidine. Glycoconj. J., 22, 63-69.
14. Xu, Z. L., Mizuguchi, H., Ishii-Watabe, A., Uchida, E., Mayumi, T., Hayakawa, T. (2001) Optimization of transcriptional regulatory elements for constructing plasmid vectors. Gene., 272, 149-156.
15. Xia, W., Bringmann, P., McClary, J., Jones, P. P., Manzana, W., Zhu, Y., Wang, S., Liu, Y., Harvey, S., Madlansacay, M. R., et al. (2006) High levels of protein expression using different mammalian CMV promoters in several cell lines. Protein Expr. Purif., 45, 115-124.
16. Al-Dosari, M., Zhang, G., Knapp, J. E., Liu, D. (2006) Evaluation of viral and mammalian promoters for driving transgene expression in mouse liver. Biochem. Biophys. Res. Commun., 339, 673-678.
17. Xu, Z. L., Mizuguchi, H., Ishii-Watabe, A., Uchida, E., Mayumi, T., Hayakawa, T. (2001) Optimization of transcriptional regulatory elements for constructing plasmid vectors. Gene., 272, 149-156.
18. Nitta, Y., Kawamoto, S., Halbert, C., Iwata, A., Miller, A. D., Miyazaki, J., Allen, M. D. (2005) A CMV-actin-globin hybrid promoter improves adeno-associated viral vector gene expression in the arterial wall in vivo. J. Gene Med., 7, 1348-1355.
19. Xia, W., Bringmann, P., McClary, J., Jones, P. P., Manzana, W., Zhu, Y., Wang, S., Liu, Y., Harvey, S., Madlansacay, M. R., et al. (2006) High levels of protein expression using different mammalian CMV promoters in several cell lines. Protein Expr. Purif., 45, 115-124.
20. Kim, S. Y., Lee, J. H., Shin, H. S., Kang, H. J., Kim, Y. S. (2002) The human elongation factor 1 alpha (EF-1 alpha) first intron highly enhances expression of foreign genes from the murine cytomegalovirus promoter. J. Biotechnol., 93, 183-187.
21. Yew, N. S., Wysokenski, D. M., Wang, K. X., Ziegler, R. J., Marshall, J., McNeilly, D., Chemy, M., Osbum, W., Cheng, S. H. (1997) Optimization of plasmid vectors for high-level expression in lung epithelial cells. Hum. Gene Ther., 8, 575-584.
22. Thomas, M. C., Chiang, C. M. (2006) The general transcription machinery and general cofactors. Crit. Rev. Biochem. Mol Biol., 41, 105-178.
23. Lim, C. Y., Santoso, B., Boulay, T., Dong, Ohler, U., Kadonaga, J. T. (2004) The MTE, a new core promoter element for transcription by RNA polymerase H. Genes Dev., 18, 1606-1617.
24. Kadonaga, J. T. (2002) The DPE, a core promoter element for transcription by RNA polymerase II. Exp. Mol. Med., 34, 259-264.
25. Smale, S. T., Kadonaga, J. T. (2003) The RNA polymerase II core promoter. Annu. Rev. Biochem., 72, 449-479.
26. Butler, J. E., Kadonaga, J. T. (2002) The RNA polymerase II core promoter: a key component in the regulation of gene expression. Genes Dev., 16, 2583-2592.
27. Butler, J. E., Kadonaga, J. T. (2001) Enhancer-promoter specificity mediated by DPE or TATA core promoter motifs. Genes Dev., 15, 2515-2519.
28. Thomas, M. C., Chiang, C. M. (2006) The general transcription machinery and general cofactors. Crit Rev. Biochem. Mol Biol., 41, 105-178.
29. Gershenzon, N.L, Ioshikhes, I. P. (2005) Synergy of human Pol II core promoter elements revealed by statistical sequence analysis. Bioinformatics., 21, 1295-1300.
30. Lim, C. Y., Santoso, B., Boulay, T., Dong, E., Ohler, U., Kadonaga, J. T. (2004) The MTE, a new core promoter element for transcription by RNA polymerase II. Genes Dev., 18, 1606-1617.
31. Gershenzon, N. I., Ioshikhes, I. P. (2005) Synergy of human Pol II core promoter elements revealed by statistical sequence analysis. Bioinformatics., 21, 1295-1300.
32. FitzGerald, P. C., Shlyakhtenko, A., Mir, A. A., Vinson, C. (2004) Clustering of DNA sequences in human promoters. Genome Res., 14, 1562-1574.
33. Lim, C. Y., Santoso, B., Boulay, T., Dong, E., Ohler, U., Kadonaga, J. T. (2004) The MTE, a new core promoter element for transcription by RNA polymerase II. Genes Dev., 18, 1606-1617.
34. Kadonaga, J. T. (2002) The DPE, a core promoter element for transcription by RNA polymerase II. Exp. Mol. Med., 34, 259-264.
35. Thomas, M. C., Chiang, C. M. (2006) The general transcription machinery and general cofactors. Crit Rev. Biochem. Mol Biol., 41, 105-178.
36. Riu, E., Chen, Z. Y., Xu, H., He, C. Y., Kay, M. A. (2007) Histone Modifications are Associated with the Persistence or Silencing of Vector-mediated Transgene Expression In Vivo. Mol Ther., 15, 1348-1355.
37. Chen, Z. Y., He, C. Y., Kay, M. A. (2005) Improved production and purification of minicircle DNA vector free of plasmid bacterial sequences and capable of persistent transgene expression in vivo. Hum. Gene Ther., 16, 126-131.
38. Chen, Z. Y., He, C. Y., Meuse, L., Kay, M. A. (2004) Silencing of episomal transgene expression by plasmid bacterial DNA elements in vivo. Gene Ther., 11, 856-864.
39. Chen, Z. Y., He, C. Y., Ehrhardt, A., Kay, M. A. (2003) Minicircle DNA vectors devoid of bacterial DNA result in persistent and high-level transgene expression in vivo. Mol Ther., 8, 495-500.

### APPENDIX A

### Promoter Sequences

### CMV Promoter Sequence (568 bp)

TAGTTATTAATAGTAATCAATTACGGGGTCATTAGTTCATAGCCCATA
TATGGAGTTCCGCGTTACATAACTTACGGTAAATGGCCCGCCTGGCTG
ACCGCCCAACGACCCCCGCCCATTGACGTCAATAATGACGTATGTTCC
CATAGTAACGCCAATAGGGACTTTCCATTGACGTCAATGGGTGGAGTA
TTTACGGTAAACTGCCCACTTGGCAGTACATCAAGTGTATCATATGCC
AAGTACGCCCCCTATTGACGTCAATGACGGTAAATGGCCCGCCTGGCA
TTATGCCCAGTACATGACCTTATGGGACTTTCCTACTTGGCAGTACAT
CTACGTATTAGTCATCGCTATTACCATGGTGATGCGGTTTTGGCAGTA
CATCAATGGGCGTGGATAGCGGTTTGACTCACGGGGATTTCCAAGTCT
CCACCCCATTGACGTCAATGGGAGTTTGTTTTGGCACCAAAATCAACG
GGACTTTCCAAAATGTCGTAACAACTCCGCCCCATTGACGCAAATGGG
CGGTAGGCGTGTACGGTGGGAGGTCTATATAAGCAGAGCT

This is human cytomegalovirus (CMV) immediate-early promoter.

### SFH Composite Promoter Sequence (716 bp)

TACGTATTAGTCATCGCTATTAACATGGGGCCTGAAATAACCTCTGAA
AGAGGAACTTGGTTAGGTACCTTCTGAGGCTGAAAGAACCAGCTGTGG
AATGTGTGTCAGTTAGGGTGTGGAAAGTCCCCAGGCTCCCCAGCAGGC
AGAAGTATGCAAAGCATGCATCTCAATTAGTCAGCAACCAGGTGTGGA
AAGTCCCCAGGCTCCCCAGCAGGCAGAAGTATGCAAAGCATGCATCTC
AATTAGTCAGCAACCATAGTCCCACTAGTTCCGCCAGAGCGCGCGAGG
GCCTCCAGCGGCCGCCCCTCCCCCACAGCAGGGGCGGGGTCCCGCGCC
CACCGGAAGGAGCGGGCTCGGGGCGGGCGGCGCTGATTGGCCGGGGCG
GGCCTGACGCCGACGCGGCTATAAGAGACCACAAGCGACCCGCAGGGC
CAGACGTTCTTCGCCGAGGCTCGCATCTCTCCTTCACGCGCCCGCCGC
CCTACCTGAGGCCGCCATCCACGCCGGTTGAGTCGCGTTCTGCCGCCT
CCCGCCTGTGGTGCCTCCTGAACTGCGTCCGCCGTCTAGGTAAGTTTA
AAGCTCAGGTCGAGACCGGGCCTTTGTCCGGCGCTCCCTTGGAGCCTA
CCTAGACTCAGCCGGCTCTCCACGCTTTGCCTGACCCTGCTTGCTCAA
CTCTACGTCTTTGTTTCGTTTTCTGTTCTGCGCCGTTACAGATC

This is a synthetic composite promoter containing SV40 enhancer, human ferritin heavy chain promoter and the human T-cell leukemia virus (HTLV) 5′ UTR.

### CAG Composite Promoter Sequence (1686 bp)

TAGTTATTAATAGTAATCAATTACGGGGTCATTAGTTCATAGCCCATA
TATGGAGTTCCGCGTTACATAACTTACGGTAAATGGCCCGCCTGGCTG
ACCGCCCAACGACCCCCGCCCATTGACGTCAATAATGACGTATGTTCC
CATAGTAACGCCAATAGGGACTTTCCATTGACGTCAATGGGTGGAGTA
TTTACGGTAAACTGCCCACTTGGCAGTACATCAAGTGTATCATATGCC
AAGTACGCCCCCTATTGACGTCAATGACGGTAAATGGCCCGCCTGGCA
TTATGCCCAGTACATGACCTTATGGGACTTTCCTACTTGGCAGTACAT
CTACGTATTAGTCATCGCTATTAACATGGTCGAGGTGAGCCCCACGTT
CTGCTTCACTCTCCCCATCTCCCCCCCCTCCCCACCCCCAATTTTGTA
TTTATTTATTTTTTAATTAITTTGTGCAGCGATGGGGGCGGGGGGGGG
GGGGGGGCGCGCGCCAGGCGGGGCGGGGCGGGGCGAGGGGCGGGGCGG
GGCGAGGCGGAGAGGTGCGGCGGCAGCCAATCAGAGCGGCGCGCTCCG
AAAGTTTCCTTTTATGGCGAGGCGGCGGCGGCGGCGGCCCTATAAAAA
GCGAAGCGCGCGGCGGGCGGGGAGTCGCTGCGACGCTGCCTTCGCCCC
GTGCCCCGCTCCGCCGCCGCCTCGCGCCGCCCGCCCCGGCTCTGACTG
ACCGCGTTACTCCCACAGGTGAGCGGGCGGGACGGCCCTTCTCCTCCG
GGCTGTAATTAGCGCTTGGTTTAATGACGGCTTGTTTCTTTTCTGTGG
CTGCGTGAAAGCCTTGAGGGGCTCCGGGAGGGCCCTTTGTGCGGGGGG
AGCGGCTCGGGGGGTGCGTGCGTGTGTGTGTGCGTGGGGAGCGCCGCG
TGCGGCTCCGCGCTGCCCGGCGGCTGTGAGCGCTGCGGGCGCGGCGCG
GGGCTTTGTGCGCTCCGCAGTGTGCGCGAGGGGAGCGCGGCCGGGGGC
GGTGCCCCGCGGTGCGGGGGGGGCTGCGAGGGGAACAAAGGCTGCGTG
CGGGGTGTGTGCGTGGGGGGGTGAGCAGGGGGTGTGGGCGCGTCGGTC
GGGCTGCAACCCCCCCTGCACCCCCCTCCCCGAGTTGCTGAGCACGGC
CCGGCTTCGGGTGCGGGGCTCCGTACGGGGCGTGGCGCGGGGCTCGCC
GTGCCGGGCGGGGGGTGGCGGCAGGTGGGGGTGCCGGGCGGGGCGGGG
CCGCCTCGGGCCGGGGAGGGCTCGGGGGAGGGGCGCGGCGGCCCCCGG
AGCGCCGGCGGCTGTCGAGGCGCGGCGAGCCGCAGCCATTGCCTTTTA
TGGTAATCGTGCGAGAGGGCGCAGGGACTTCCTTTGTCCCAAATCTGT
GCGGAGCCGAAATCTGGGAGGCGCCGCCGCACCCCCTCTAGCGGGCGC
GGGGCGAAGCGGTGCGGCGCCGGCAGGAAGGAAATGGGCGGGGAGGGC
CTTCGTGCGTCGCCGCGCCGCCGTCCCCTTCTCCCTCTCCAGCCTCGG
GGCTGTCCGCGGGGGGACGGCTGCCTTCGGGGGGGACGGGGCAGGGCG
GGGTTCGGCTTCTGGCGTGTGACCGGCGGCTCTAGACAATTGTACTAA
CCTTCTTCTCTTTCCTCTCCTGACAGGTTGGTGTACAGTAGCTTCCAC
GAGCTC

This is a synthetic composite promoter containing human CMV enhancer, modified chicken beta-actin promoter and its first intron.

### CEFR Composite Promoter Sequence (936 bp)

TAGTTATTAATagatcTTAATAGTAATCAATTACGGGGTCATTAGTTC
ATAGCCCATATATGGAGTTCCGCGTTACATAACTTACGGTAAATGGCC
CGCCTGGCTGACCGCCCAACGACCCCCGCCCATTGACGTCAATAATGA
CGTATGTTCCCATAGTAACGCCAATAGGGACTTTCCATTGACGTCAAT
GGGTGGAGTATTTACGGTAAACTGCCCACTTGGCAGTACATCAAGTGT
ATCATATGCCAAGTACGCCCCCTATTGACGTCAATGACGGTAAATGGC
CCGCCTGGCATTATGCCCAGTACATGACCTTATGGGACTTTCCTACTT
GGCAGTACATCTACGTATTAGTCATCGCTATTAACATggatcTGGATC
TGCGATCGCTCCGGTGCCCGTCAGTGGGCAGAGCGCACATCGCCCACA
GTCCCCGAGAAGTTGGGGGGAGGGGTCGGCAATTGAACCGGTGCCTAG
AGAAGGTGGCGCGGGGTAAACTGGGAAAGTGATGTCGTGTACTGGCTC
CGCCTTTTTCCCGAGGGTGGGGGAGAACCGTATATAAGTGCAGTAGTC
GCCGTGAACGTTCTTTTTCGCAACGGGTTTGCCGCCAGAACACAGCTG
AAGCTTCGAGGGGCTCGCATCTCTCCTTCACGCGCCCGCCGCCCTACC
TGAGGCCGCCATCCACGCCGGTTGAGTCGCGTTCTGCCGCCTCCCGCC
TGTGGTGCCTCCTGAACTGCGTCCGCCGTCTAGGTAAGTTTAAAGCTC
AGGTCGAGACCGGGCCTTTGTCCGGCGCTCCCTTGGAGCCTACCTAGA
CTCAGCCGGCTCTCCACGCTTTGCCTGACCCTGCTTGCTCAACTCTAC
GTCTTTGTTTCGTTTTCTGTTCTGCGCCGTTACAGATCCAAGCTGTGA
CCGGCGCCTACCTGAGATGAGCTC

This is a synthetic composite promoter containing human CMV enhancer, the elongation factor EF1α core promoter, and HTLV 5′ UTR.

### EFHT Composite Promoter Sequence (576 bp)

GATTAATAGATCTGGATCTGGATCTGCGATCGCTCCGGTGCCCGTCAG
TGGGCAGAGCGCACATCGCCCACAGTCCCCGAGAAGTTGGGGGGAGGG
GTCGGCAATTGAACCGGTGCCTAGAGAAGGTGGCGCGGGGTAAACTGG
GAAAGTGATGTCGTGTACTGGCTCCGCCTTTTTCCCGAGGGTGGGGGA
GAACCGTATATAAGTGCAGTAGTCGCCGTGAACGTTCTTTTTCGCAAC
GGGTTTGCCGCCAGAACACAGCTGAAGCTTCGAGGGGCTCGCATCTCT
CCTTCACGCGCCCGCCGCCCTACCTGAGGCCGCCATCCACGCCGGTTG
AGTCGCGTTCTGCCGCCTCCCGCCTGTGGTGCCTCCTGAACTGCGTCC
GCCGTCTAGGTAAGTTTAAAGCTCAGGTCGAGACCGGGCCTTTGTCCG
GCGCTCCCTTGGAGCCTACCTAGACTCAGCCGGCTCTCCACGCTTTGC
CTGACCCTGCTTGCTCAACTCTACGTCTTTGTTTCGTTTTCTGTTCTG
CGCCGTTACAGATCCAAGCTGTGACCGGCGCCTACCTGAGATGAGCTC

This is a synthetic composite promoter containing the elongation factor EF1α core promoter and HTLV 5′ UTR.

### APPENDIX B

### Core Promoter 0 Sequence (C0, 87 nt)

GAGCTCTCTGGCTAACTAGAGAACCCACTGCTTACTGGCTTATCGAAA
TTAATACGACTCACTATAGGGAGACCCAAGCTGGCTAGC

### Core Promoter 1 Sequence (C1, 88 nt)

GAGCTCGGACGCCTATATAAGCAGAGCTGGTTTAGTGAACCGTCAGTT
AATACGACTCACTATAGGCGAACGCAACGGACGTGCTAGC

### Core Promoter 2 Sequence (C2, 56 nt)

GAGCTCGGACGCCTATATAAGCAGAGCTGGTTTAGTGAACCGTCAGAT
CCGCTAGC

### Core Promoter 3 Sequence (C3, 83 nt)

GAGCTCGCAAATGGGCGGTAGGCGTGTACGGTGGGAGGTCTATATAAG
CAGAGCTGGTTTAGTGAACCGTCAGATCCGCTAGC

### APPENDIX C

### FMDV IRES Sequence (469 nt)

AGCAGGTTTCCCCAATGACACAAAACGTGCAACTTGAAACTCCGCCTG
GTCTTTCCAGGTCTAGAGGGGTAACACTTTGTACTGCGTTTGGCTCCA
CGCTCGATCCACTGGCGAGTGTTAGTAACAGCACTGTTGCTTCGTAGC
GGAGCATGACGGCCGTGGGAACTCCTCCTTGGTAACAAGGACCCACGG
GGCCAAAAGCCACGCCCACACGGGCCCGTCATGTGTGCAACCCCAGCA
CGGCGACTTTACTGCGAAACCCACTTTAAAGTGACATTGAAACTGGTA
CCCACACACTGGTGACAGGCTAAGGATGCCCTTCAGGTACCCCGAGGT
AACACGCGACACTCGGGATCTGAGAAGGGGACTGGGGCTTCTATAAAA
GCGCTCGGTTTAAAAAGCTTCTATGCCTGAATAGGTGACCGGAGGTCG
GCACCTTTCCTTTGCAATTACTGACCCTATGAATACA

### Synthetic GTX IRES Sequence (179 nt)

CCGGCGGGTTTCTGACATCCGGCGGGTTTCTGACATCCGGCGGGTTTC
TGACATCCGGCGGGTTTCTGACATCCGGCGGGTGATTAATTCCTTCTG
ACATCCGGCGGGTTTCTTACACCGGCGGTTTCTGACATCCGGCGGGTT
TCTGACATCCGGCGGGTTTCTGACATCCGGCGGGT

### APPENDIX D

### Synthetic MAR-Q Sequence (903 nt)

AGATCCCCAGCTCCATTATTATTTATGTGAATATTCATCCAATTGACG
TCCATTTTTTTTGCTGACTTCTGAATATATTTTTTAATTTCACTTGTA
AAACTTTGGTGTGTTTCGATATGGTTTGATTTATATAATAATATATTT
TTTAATTTCACTTGTAAAACTTTGGGTGTGTTTAATTTATTTATCCAG
CTCCAAGATCCCCAGCTCCATTATTATTTATGTGAATATTCATCCAAT
TGACGTCCATTTTTTTTGCTGACTTCTGAATATATTTTTTAATTTCAC
TTGTAAAACTTTGGTGTGTTTCGATATGGTTTGATTTATATAATAATA
TATTTTTTAATTTCACTTGTAAAACTTTGGGTGTGTTTAATTTATTTA
TCCAGCTCCAAGATCTAGGTGCAGAGTGATTTGCCGTGGTGGCTGGTC
TGCCGGGGGACGATTCATAAGTTCCGCTGTGTGCCGCATCTCACAGCA
GATCTGTTTTACAAGTGAAATTAAAAAATATATTGAAGTCAGCCATTT
TTAAAAATTTGATTGGATGAATATTCATTTGGAGCTGGCATATCGCAC
ATGGACGTCACTAAATATATTAGAATATATAAATCAAATAAATTATCG
CACAATAATAAATTATATATAAATATATACTAAGTTTAAATGGATCTG
TTTTACAAGTGAAATTAAAAAATATATTGAAGTCAGCCATTTTTAAAA
ATTTGATTGGATGAATATTCATTTGGAGCTGGCATATCGCACATGGAC
GTCACTAAATATATTAGAATATATAAATCAAATAAATTATCGCACAAT
AATAAATTATATATAAATATATACTAAGTTTAAATGGATCTTAATAGT
AATCAATTACGGGGTCATTAGTCATAGCCCAATAGATCC

### Synthetic MAR-R Sequence (575 nt)

AGATCCCCAGCTCCATTATTATTTATGTGAATATTCATCCAATTGACG
TCCATTTTTTTTGCTGACTTCTGAATATATTTTTTAATTTCACTTGTA
AAACTTTGGTGTGTTTCGATATGGTTTGATTTATATAATAATATATTT
TTTAATTTCACTTGTAAAACTTTGGGTGTGTTTAATTTATTTATCCAG
CTCCAAGATCCCCAGCTCCATTATTATTTATGTGAATATTCATCCAAT
TGACGTCCATTTTTTTTGCTGACTTCTGAATATATTTTTTAATTTCAC
TTGTAAAACTTTGGTGTGTTTCGATATGGTTTGATTTATATAATAATA
TATTTTTTAATTTCACTTGTAAAACTTTGGGTGTGTTTAATTTATTTA
TCCAGCTTTTTACAAGTGAAATTAAAAAATATATTGAAGTCAGCCATT
TTTAAAAATTTGATTGGATGAATATTCATTTGGAGCTGGCATATCGCA
CATGGACGTCACTAAATATATTAGAATATATAAATCAAATAAATTATC
GCACAATAATAAATTATATATAAATATATACTAAGTTTAAATGGATC

### Synthetic MAR-S Sequence (758 nt)

TCTTTAATTTCTAATATATTTAGAAGGCATGCTTCTATATTATTTTCT
AAAAGATTTAAAGTTTTGCCTTCTCCATTTAGACTTATAATTCACTGG
AATTTTTTTGTGTGTATGGTATGAGATATGGGTTCCCTTTTATTTTTT
ACATATAAATATATTTCCCTGTTTTTCTAAAAAAGAGTTTGATTTATA
TATATTTAAACTTAGTATATATTTATATATAATTTATTATTGTGCGAT
AATTTAGTTTGATTTATATATTCTAATATATTTAGTGACGTCCATGTG
CGATATGCCAGCTCCAAATGAATATTCATCCAATCAAATTTTTAAAAA
TGGCTGACTTCAATATATTTTTTAATTTCACTTGTAAAACAGATCCAT
TTAAACTTAGTATATATTTATATATAATTTATTATTGTGCGATAATTT
AGTTTGATTTATATATTCTAATATATTTAGTGACGTCCATGTGCGATA
TGCCAGCTCCAAATGAATATTCATCCAATCAAATTTTTAAAAATGGCT
GACTTCAATATATTTTTTAATTTCACTTGTAAAACTCTTTAATTTCTA
ATATATTTAGAAGGCATGCTTCTATATTATTTTCTAAAAGATTTAAAG
TTTTGCCTTCTCCATTTAGACTTATAATTCACTGGAATTTTTTTGTGT
GTATGGTATGAGATATGGGTTCCCTTTTATTTTTTACATATAAATATA
TTTCCCTGTTTTTCTAAAAAAGAGTTTGATTTATATAT

### APPENDIX E

### Composite Selective Marker Genes

### ZEO-PUR Composite Gene Sequence (984 nt)

ATGGCCAAGTTGACCAGTGCCGTTCCGGTGCTCACCGCGCGCGACGTC
GCCGGAGCGGTCGAGTTCTGGACCGACCGGCTCGGGTTCTCCCGGGAC
TTCGTGGAGGACGACTTCGCCGGTGTGGTCCGGGACGACGTGACCCTG
TTCATCAGCGCGGTCCAGGACCAGGTGGTGCCGGACAACACCCTGGCC
TGGGTGTGGGTGCGCGGCCTGGACGAGCTGTACGCCGAGTGGTCGGAG
GTCGTGTCCACGAACTTCCGGGACGCCTCCGGGCCGGCCATGACCGAG
ATCGGCGAGCAGCCGTGGGGGCGGGAGTTCGCCCTGCGCGACCCGGCC
GGCAACTGCGTGCACTTCGTGGCCGAGGAGCAGGACGATACTATGACC
GAGTACAAGCCCACGGTGCGCCTCGCCACCCGCGACGACGTCCCCAGG
GCCGTACGCACCCTCGCCGCCGCGTTCGCCGACTACCCCGCCACGCGC
CACACCGTCGATCCGGACCGCCACATCGAGCGGGTCACCGAGCTGCAA
GAACTCTTCCTCACGCGCGTCGGGCTCGACATCGGCAAGGTGTGGGTC
GCGGACGACGGCGCCGCGGTGGCGGTCTGGACCACGCCGGAGAGCGTC
GAAGCGGGGGCGGTGTTCGCCGAGATCGGCCCGCGCATGGCCGAGTTG
AGCGGTTCCCGGCTGGCCGCGCAGCAACAGATGGAAGGCCTCCTGGCG
CCGCACCGGCCCAAGGAGCCCGCGTGGTTCCTGGCCACCGTCGGCGTC
TCGCCCGACCACCAGGGCAAGGGTCTGGGCAGCGCCGTCGTGCTCCCC
GGAGTGGAGGCGGCCGAGCGCGCCGGGGTGCCCGCCTTCCTGGAGACC
TCCGCGCCCCGCAACCTCCCCTTCTACGAGCGGCTCGGCTTCACCGTC
ACCGCCGACGTCGAGGTGCCCGAAGGACCGCGCACCTGGTGCATGACC
CGCAAGCCCGGTGCCTCGATCTGA

### ZEO-HYG Composite Gene Sequence (1404 nt)

ATGGCCAAGTTGACCAGTGCCGTTCCGGTGCTCACCGCGCGCGACGTC
GCCGGAGCGGTCGAGTTCTGGACCGACCGGCTCGGGTTCTCCCGGGAC
TTCGTGGAGGACGACTTCGCCGGTGTGGTCCGGGACGACGTGACCCTG
TTCATCAGCGCGGTCCAGGACCAGGTGGTGCCGGACAACACCCTGGCC
TGGGTGTGGGTGCGCGGCCTGGACGAGCTGTACGCCGAGTGGTCGGAG
GTCGTGTCCACGAACTTCCGGGACGCCTCCGGGCCGGCCATGACCGAG
ATCGGCGAGCAGCCGTGGGGGCGGGAGTTCGCCCTGCGCGACCCGGCC
GGCAACTGCGTGCACTTCGTGGCCGAGGAGCAGGACGATACTATGAAG
AAACCTGAACTGACAGCAACTTCTGTTGAGAAGTTTCTCATTGAAAAA
TTTGATTCTGTTTCTGATCTCATGCAGCTGTCTGAAGGTGAAGAAAGC
AGAGCCTTTTCTTTTGATGTTGGAGGAAGAGGTTATGTTCTGAGGGTC
AATTCTTGTGCTGATGGTTTTTACAAAGACAGATATGTTTACAGACAC
TTTGCCTCTGCTGCTCTGCCAATTCCAGAAGTTCTGGACATTGGAGAA
TTTTCTGAATCTCTCACCTACTGCATCAGCAGAAGAGCACAAGGAGTC
ACTCTCCAGGATCTCCCTGAAACTGAGCTGCCAGCTGTTCTGCAACCT
GTTGCTGAAGCAATGGATGCCATTGCAGCAGCTGATCTGAGCCAAACC
TCTGGATTTGGTCCTTTTGGTCCCCAAGGCATTGGTCAGTACACCACT
TGGAGGGATTTCATTTGTGCCATTGCTGATCCTCATGTCTATCACTGG
CAGACTGTGATGGATGACACAGTTTCTGCTTCTGTTGCTCAGGCACTG
GATGAACTCATGCTGTGGGCAGAAGATTGTCCTGAAGTCAGACACCTG
GTCCATGCTGATTTTGGAAGCAACAATGTTCTGACAGACAATGGCAGA
ATCACTGCAGTCATTGACTGGTCTGAAGCCATGTTTGGAGATTCTCAA
TATGAGGTTGCCAACATTTTTTTTTGGAGACCTTGGCTGGCTTGCATG
GAACAACAAACAAGATATTTTGAAAGAAGACACCCAGAACTGGCTGGT
TCCCCCAGACTGAGAGCCTACATGCTCAGAATTGGCCTGGACCAACTG
TATCAATCTCTGGTTGATGGAAACTTTGATGATGCTGCTTGGGCACAA
GGAAGATGTGATGCCATTGTGAGGTCTGGTGCTGGAACTGTTGGAAGA
ACTCAAATTGCAAGAAGGTCTGCTGCTGTTTGGACTGATGGATGTGTT
GAAGTTCTGGCTGACTCTGGAAACAGGAGACCCTCCACAAGACCCAGA
GCCAAGGAATGA

### ZEO-GFP Composite Gene Sequence (1098 nt)

ATGGCCAAGTTGACCAGTGCCGTTCCGGTGCTCACCGCGCGCGACGTC
GCCGGAGCGGTCGAGTTCTGGACCGACCGGCTCGGGTTCTCCCGGGAC
TTCGTGGAGGACGACTTCGCCGGTGTGGTCCGGGACGACGTGACCCTG
TTCATCAGCGCGGTCCAGGACCAGGTGGTGCCGGACAACACCCTGGCC
TGGGTGTGGGTGCGCGGCCTGGACGAGCTGTACGCCGAGTGGTCGGAG
GTCGTGTCCACGAACTTCCGGGACGCCTCCGGGCCGGCCATGACCGAG
ATCGGCGAGCAGCCGTGGGGGCGGGAGTTCGCCCTGCGCGACCCGGCC
GGCAACTGCGTGCACTTCGTGGCCGAGGAGCAGGACGATCCTATGGTG
AGCAAGGGCGAGGAGCTGTTCACCGGGGTGGTGCCCATCCTGGTCGAG
CTGGACGGCGACGTAAACGGCCACAAGTTCAGCGTGTCCGGCGAGGGC
GAGGGCGATGCCACCTACGGCAAGCTGACCCTGAAGTTCATCTGCACC
ACCGGCAAGCTGCCCGTGCCCTGGCCCACCCTCGTGACCACCCTGACC
TACGGCGTGCAGTGCTTCAGCCGCTACCCCGACCACATGAAGCAGCAC
GACTTCTTCAAGTCCGCCATGCCCGAAGGCTACGTCCAGGAGCGCACC
ATCTTCTTCAAGGACGACGGCAACTACAAGACCCGCGCCGAGGTGAAG
TTCGAGGGCGACACCCTGGTGAACCGCATCGAGCTGAAGGGCATCGAC
TTCAAGGAGGACGGCAACATCCTGGGGCACAAGCTGGAGTACAACTAC
AACAGCCACAACGTCTATATCATGGCCGACAAGCAGAAGAACGGCATC
AAGGTGAACTTCAAGATCCGCCACAACATCGAGGACGGCAGCGTGCAG
CTCGCCGACCACTACCAGCAGAACACCCCCATCGGCGACGGCCCCGTG
CTGCTGCCCGACAACCACTACCTGAGCACCCAGTCCGCCCTGAGCAAA
GACCCCAACGAGAAGCGCGATCACATGGTCCTGCTGGAGTTCGTGACC
GCCGCCGGGATCACTCTCGGCATGGACGAGCTGTACAAGTAG

### DHFR-ZEO-PUR Composite Gene Sequence (1551 nt)

ATGGTTGGTTCGCTAAACTGCATCGTCGCTGTGTCCCAGAGCATGGGC
ATCGGCAAGAACGGGGACCTGCCCTGGCCACCGCTCAGGAATGAATTC
AGATATTTCCAGAGAATGACCACAACCTCTTCAGTAGAAGGTAAACAG
AATCTGGTGATTATGGGTAAGAAGACCTGGTTCTCCATTCCTGAGAAG
AATCGACCTTTAAAGGGTAGAATTAATTTAGTTCTCAGCAGAGAACTC
AAGGAACCTCCACAAGGAGCTCATTTTCTTTCCAGAAGTCTAGATGAT
GCCTTAAAACTTACTGAACAACCAGAATTAGCAAATAAAGTAGACATG
GTCTGGATAGTTGGTGGCAGTTCTGTTTATAAGGAAGCCATGAATCAC
CCAGGCCATCTTAAACTATTTGTGACAAGGATCATGCAAGACTTTGAA
AGTGACACGTTTTTTCCAGAAATTGATTTGGAGAAATATAAACTTCTG
CCAGAATACCCAGGTGTTCTCTCTGATGTCCAGGAGGAGAAAGGCATT
AAGTACAAATTTGAAGTATATGAGAAGAATGATGATACTATGGCCAAG
TTGACCAGTGCCGTTCCGGTGCTCACCGCGCGCGACGTCGCCGGAGCG
GTCGAGTTCTGGACCGACCGGCTCGGGTTCTCCCGGGACTTCGTGGAG
GACGACTTCGCCGGTGTGGTCCGGGACGACGTGACCCTGTTCATCAGC
GCGGTCCAGGACCAGGTGGTGCCGGACAACACCCTGGCCTGGGTGTGG
GTGCGCGGCCTGGACGAGCTGTACGCCGAGTGGTCGGAGGTCGTGTCC
ACGAACTTCCGGGACGCCTCCGGGCCGGCCATGACCGAGATCGGCGAG
CAGCCGTGGGGGCGGGAGTTCGCCCTGCGCGACCCGGCCGGCAACTGC
GTGCACTTCGTGGCCGAGGAGCAGGACGATACTATGACCGAGTACAAG
CCCACGGTGCGCCTCGCCACCCGCGACGACGTCCCCAGGGCCGTACGC
ACCCTCGCCGCCGCGTTCGCCGACTACCCCGCCACGCGCCACACCGTC
GATCCGGACCGCCACATCGAGCGGGTCACCGAGCTGCAAGAACTCTTC
CTCACGCGCGTCGGGCTCGACATCGGCAAGGTGTGGGTCGCGGACGAC
GGCGCCGCGGTGGCGGTCTGGACCACGCCGGAGAGCGTCGAAGCGGGG
GCGGTGTTCGCCGAGATCGGCCCGCGCATGGCCGAGTTGAGCGGTTCC
CGGCTGGCCGCGCAGCAACAGATGGAAGGCCTCCTGGCGCCGCACCGG
CCCAAGGAGCCCGCGTGGTTCCTGGCCACCGTCGGCGTCTCGCCCGAC
CACCAGGGCAAGGGTCTGGGCAGCGCCGTCGTGCTCCCCGGAGTGGAG
GCGGCCGAGCGCGCCGGGGTGCCCGCCTTCCTGGAGACCTCCGCGCCC
CGCAACCTCCCCTTCTACGAGCGGCTCGGCTTCACCGTCACCGCCGAC
GTCGAGGTGCCCGAAGGACCGCGCACCTGGTGCATGACCCGCAAGCCC
GGTGCCTCGATCTGA

### DHFR-ZEO-GFP Composite Gene Sequence (1665 nt)

ATGGTTGGTTCGCTAAACTGCATCGTCGCTGTGTCCCAGAGCATGGGC
ATCGGCAAGAACGGGGACCTGCCCTGGCCACCGCTCAGGAATGAATTC
AGATATTTCCAGAGAATGACCACAACCTCTTCAGTAGAAGGTAAACAG
AATCTGGTGATTATGGGTAAGAAGACCTGGTTCTCCATTCCTGAGAAG
AATCGACCTTTAAAGGGTAGAATTAATTTAGTTCTCAGCAGAGAACTC
AAGGAACCTCCACAAGGAGCTCATTTTCTTTCCAGAAGTCTAGATGAT
GCCTTAAAACTTACTGAACAACCAGAATTAGCAAATAAAGTAGACATG
GTCTGGATAGTTGGTGGCAGTTCTGTTTATAAGGAAGCCATGAATCAC
CCAGGCCATCTTAAACTATTTGTGACAAGGATCATGCAAGACTTTGAA
AGTGACACGTTTTTTCCAGAAATTGATTTGGAGAAATATAAACTTCTG
CCAGAATACCCAGGTGTTCTCTCTGATGTCCAGGAGGAGAAAGGCATT
AAGTACAAATTTGAAGTATATGAGAAGAATGATGATACTATGGCCAAG
TTGACCAGTGCCGTTCCGGTGCTCACCGCGCGCGACGTCGCCGGAGCG
GTCGAGTTCTGGACCGACCGGCTCGGGTTCTCCCGGGACTTCGTGGAG
GACGACTTCGCCGGTGTGGTCCGGGACGACGTGACCCTGTTCATCAGC
GCGGTCCAGGACCAGGTGGTGCCGGACAACACCCTGGCCTGGGTGTGG
GTGCGCGGCCTGGACGAGCTGTACGCCGAGTGGTCGGAGGTCGTGTCC
ACGAACTTCCGGGACGCCTCCGGGCCGGCCATGACCGAGATCGGCGAG
CAGCCGTGGGGGCGGGAGTTCGCCCTGCGCGACCCGGCCGGCAACTGC
GTGCACTTCGTGGCCGAGGAGCAGGACGATCCTATGGTGAGCAAGGGC
GAGGAGCTGTTCACCGGGGTGGTGCCCATCCTGGTCGAGCTGGACGGC
GACGTAAACGGCCACAAGTTCAGCGTGTCCGGCGAGGGCGAGGGCGAT
GCCACCTACGGCAAGCTGACCCTGAAGTTCATCTGCACCACCGGCAAG
CTGCCCGTGCCCTGGCCCACCCTCGTGACCACCCTGACCTACGGCGTG
CAGTGCTTCAGCCGCTACCCCGACCACATGAAGCAGCACGACTTCTTC
AAGTCCGCCATGCCCGAAGGCTACGTCCAGGAGCGCACCATCTTCTTC
AAGGACGACGGCAACTACAAGACCCGCGCCGAGGTGAAGTTCGAGGGC
GACACCCTGGTGAACCGCATCGAGCTGAAGGGCATCGACTTCAAGGAG
GACGGCAACATCCTGGGGCACAAGCTGGAGTACAACTACAACAGCCAC
AACGTCTATATCATGGCCGACAAGCAGAAGAACGGCATCAAGGTGAAC
TTCAAGATCCGCCACAACATCGAGGACGGCAGCGTGCAGCTCGCCGAC
CACTACCAGCAGAACACCCCCATCGGCGACGGCCCCGTGCTGCTGCCC
GACAACCACTACCTGAGCACCCAGTCCGCCCTGAGCAAAGACCCCAAC
GAGAAGCGCGATCACATGGTCCTGCTGGAGTTCGTGACCGCCGCCGGG
ATCACTCTCGGCATGGACGAGCTGTACAAGTAG

### DHFR-ZEO-HYG Composite Gene Sequence (1971 nt)

ATGGTTGGTTCGCTAAACTGCATCGTCGCTGTGTCCCAGAGCATGGGC
ATCGGCAAGAACGGGGACCTGCCCTGGCCACCGCTCAGGAATGAATTC
AGATATTTCCAGAGAATGACCACAACCTCTTCAGTAGAAGGTAAACAG
AATCTGGTGATTATGGGTAAGAAGACCTGGTTCTCCATTCCTGAGAAG
AATCGACCTTTAAAGGGTAGAATTAATTTAGTTCTCAGCAGAGAACTC
AAGGAACCTCCACAAGGAGCTCATTTTCTTTCCAGAAGTCTAGATGAT
GCCTTAAAACTTACTGAACAACCAGAATTAGCAAATAAAGTAGACATG
GTCTGGATAGTTGGTGGCAGTTCTGTTTATAAGGAAGCCATGAATCAC
CCAGGCCATCTTAAACTATTTGTGACAAGGATCATGCAAGACTTTGAA
AGTGACACGTTTTTTCCAGAAATTGATTTGGAGAAATATAAACTTCTG
CCAGAATACCCAGGTGTTCTCTCTGATGTCCAGGAGGAGAAAGGCATT
AAGTACAAATTTGAAGTATATGAGAAGAATGATGATACTATGGCCAAG
TTGACCAGTGCCGTTCCGGTGCTCACCGCGCGCGACGTCGCCGGAGCG
GTCGAGTTCTGGACCGACCGGCTCGGGTTCTCCCGGGACTTCGTGGAG
GACGACTTCGCCGGTGTGGTCCGGGACGACGTGACCCTGTTCATCAGC
GCGGTCCAGGACCAGGTGGTGCCGGACAACACCCTGGCCTGGGTGTGG
GTGCGCGGCCTGGACGAGCTGTACGCCGAGTGGTCGGAGGTCGTGTCC
ACGAACTTCCGGGACGCCTCCGGGCCGGCCATGACCGAGATCGGCGAG
CAGCCGTGGGGGCGGGAGTTCGCCCTGCGCGACCCGGCCGGCAACTGC
GTGCACTTCGTGGCCGAGGAGCAGGACGATACTATGAAGAAACCTGAA
CTGACAGCAACTTCTGTTGAGAAGTTTCTCATTGAAAAATTTGATTCT
GTTTCTGATCTCATGCAGCTGTCTGAAGGTGAAGAAAGCAGAGCCTTT
TCTTTTGATGTTGGAGGAAGAGGTTATGTTCTGAGGGTCAATTCTTGT
GCTGATGGTTTTTACAAAGACAGATATGTTTACAGACACTTTGCCTCT
GCTGCTCTGCCAATTCCAGAAGTTCTGGACATTGGAGAATTTTCTGAA
TCTCTCACCTACTGCATCAGCAGAAGAGCACAAGGAGTCACTCTCCAG
GATCTCCCTGAAACTGAGCTGCCAGCTGTTCTGCAACCTGTTGCTGAA
GCAATGGATGCCATTGCAGCAGCTGATCTGAGCCAAACCTCTGGATTT
GGTCCTTTTGGTCCCCAAGGCATTGGTCAGTACACCACTTGGAGGGAT
TTCATTTGTGCCATTGCTGATCCTCATGTCTATCACTGGCAGACTGTG
ATGGATGACACAGTTTCTGCTTCTGTTGCTCAGGCACTGGATGAACTC
ATGCTGTGGGCAGAAGATTGTCCTGAAGTCAGACACCTGGTCCATGCT
GATTTTGGAAGCAACAATGTTCTGACAGACAATGGCAGAATCACTGCA
GTCATTGACTGGTCTGAAGCCATGTTTGGAGATTCTCAATATGAGGTT
GCCAACATTTTTTTTTGGAGACCTTGGCTGGCTTGCATGGAACAACAA
ACAAGATATTTTGAAAGAAGACACCCAGAACTGGCTGGTTCCCCCAGA
CTGAGAGCCTACATGCTCAGAATTGGCCTGGACCAACTGTATCAATCT
CTGGTTGATGGAAACTTTGATGATGCTGCTTGGGCACAAGGAAGATGT
GATGCCATTGTGAGGTCTGGTGCTGGAACTGTTGGAAGAACTCAAATT
GCAAGAAGGTCTGCTGCTGTTTGGACTGATGGATGTGTTGAAGTTCTG
GCTGACTCTGGAAACAGGAGACCCTCCACAAGACCCAGAGCCAAGGAA
TGA

What is claimed is:

1. A mammalian expression vector comprising a target cDNA and at least one selective marker gene linked for expression in a single mammalian expression cassette under the control of a composite promoter, wherein the target cDNA and the at least one marker gene are linked by an IRES element.

2. The mammalian expression vector of claim 1 comprising: (a) a composite promoter; (b) a core promoter; (c) at least one cloning site for target gene insertion; (d) an IRES; (e) bacterial expression promoter EM7; (f) a selective marker gene; (g) a transcription terminator; (h) PUC origin for high-copy amplification in bacteria; and (i) at least one artificial nuclear matrix attachment region (MAR) element.

3. The expression vector of claim 1 or 2 wherein the composite promoter sequence comprises a CAG promoter or a SFH promoter.

4. The expression vector of claim 3 wherein the composite promoter sequence comprises the 1686 nt CAG promoter or the 716 nt SFH promoter shown in Appendix A.

5. The expression vector of claim 2 wherein the core promoter comprises a 56 nt promoter C2 shown in Appendix B.

6. The expression vector of claim 2 wherein the IRES comprises a synthetic GTX IRES or an FMDV IRES.

7. The expression vector of claim 6 wherein the IRES comprises the synthetic 179 nt GTX IRES shown in Appendix C.

8. The expression vector of claim 1 or 2 wherein the selective marker gene comprises a composite selective marker gene.

9. The expression vector of claim 8 wherein the composite selective marker gene comprises a ZEO-PUR, ZEO-HYG, ZEO-GFP, DHFR-ZEO-PUR, DHFR-ZEO-GFP, or DHFR-ZEO-HYG fusion gene.

10. The expression vector of claim 2 wherein the at least one artificial MAR element comprises MAR-Q, MAR-R or MAR-S.

11. The expression vector of claim 1 which comprises one of REEMAC1, REEMAC2, REEMAC3, REEMAC4, REEMAC 5 or REEMAC6.

12. The expression vector of claim 2 which does not comprise a reluctant gene sequence which is not required for expression in host mammalian cells.

13. A method of expressing a target gene in a mammalian cell, utilizing an expression vector which physically links the target gene with a selective marker gene, and comprising the steps of:
(a) driving the target gene by a composite promoter;
(b) driving a selective marker gene by an IRES so as to exhibit an activity level of the selective marker gene substantially below that of its corresponding wild type.

14. The method of claim 13 wherein the vector comprises an artificial MAR element.

15. The method of claim 13 wherein the target gene is driven by a composite promoter and a core promoter.

16. The method of claim 13 wherein the IRES comprises a synthetic IRES.

9. The expression vector of claim 8 wherein the composite selective marker gene comprises a ZEO-PUR, ZEO-HYG, ZEO-GFP, DHFR-ZEO-PUR, DHFR-ZEO-GFP, or DHFR-ZEO-HYG fusion gene.

10. The expression vector of claim 2 wherein the at least one artificial MAR element comprises MAR-Q, MAR-R or MAR-S.

11. The expression vector of claim 1 which comprises one of REEMAC1, REEMAC2, REEMAC3, REEMAC4, REEMAC 5 or REEMAC6.

12. The expression vector of claim 2 which does not comprise a reluctant gene sequence which is not required for expression in host mammalian cells.

13. A method of expressing a target gene in a mammalian cell, utilizing an expression vector which physically links the target gene with a selective marker gene, and comprising the steps of:
(a) driving the target gene by a composite promoter;
(b) driving a selective marker gene by an IRES so as to exhibit an activity level of the selective marker gene substantially below that of its corresponding wild type.

14. The method of claim 13 wherein the vector comprises an artificial MAR element.

15. The method of claim 13 wherein the target gene is driven by a composite promoter and a core promoter.

16. The method of claim 13 wherein the IRES comprises a synthetic IRES.
